SEQUENCE LISTING (64 sequences)

SEQ ID NO: 1 - RNA, 80 nt, Artificial Sequence
  TMirt877v3.1
guagaggagauggcgcaggguucaagagacccugcgccaucuccuuucucaguuuggauugucucuuucuuucuucuaag

SEQ ID NO: 2 - RNA, 72 nt, Artificial Sequence
  TMirt877v3.2
guagaggagauggcgcaggguucaagagacccugcgccaucuccuuucucaguuuuuucucuuucuuucaag

SEQ ID NO: 3 - RNA, 74 nt, Artificial Sequence
  DMPKTMirt5
guaggaguagcccacaaaagguucaagagaccuuuugugggcuacucuucucaguuuuuucucuuucuuucaag

SEQ ID NO: 4 - RNA, 74 nt, Artificial Sequence
  DMPKTMirt13
guguagugaagaugaaguggguucaagagaucugcuucaucuucacuaucucaguuuuuucucuuucuuucaag

SEQ ID NO: 5 - RNA, 74 nt, Artificial Sequence
  TMirtFL19
gugagagaagcgcacacaggcuucaagagagccugugugcgcuucucuucucaguuuuuucucuuucuuucaag

SEQ ID NO: 6 - RNA, 71 nt, Artificial Sequence
  TMirtVegfa1
guaaauguauguauguggguguucaagagacacccacacacauacaucucaguuuuuucucuuucuuucag

SEQ ID NO: 7 - RNA, 73 nt, Artificial Sequence
  TMirtVegfa4
guagagcagcaaggcaaggcuuucaagagaagccuugccuugcugcucucucaguuuuuucucuuucuuucag

SEQ ID NO: 8 - RNA, 73 nt, Artificial Sequence
  TMirtVegfa7
guggaucaaaccucacuaauauucaagagauauuggugagguuugauccgucaguuuuuucucuuucuuucag

SEQ ID NO: 9 - RNA, 71 nt, Artificial Sequence
  TMirtVegfa8
guagauuaugcggauuaaauuucaagagaguuugauccgcauaaucugucaguuuuuucucuuucuuucag

SEQ ID NO: 10 - RNA, 73 nt, Artificial Sequence
  TMIrtTerC2
gugagaaccgaguuccggguguucaagagacacccggaacucgguucuucucaguuuuuucucuuucuuucag

SEQ ID NO: 11 - RNA, 73 nt, Artificial Sequence
  TMirtTerC4
guaagacaccgaacacggggauucaagagauccccguguucggugucuucucaguuuuuucucuuucuuucag

SEQ ID NO: 12 - RNA, 72 nt, Artificial Sequence
  TMirtTert7
guagaugacagaaugagcagcuucaagagagcugcucauucugucauucucaguuuuuucucuuucuuucag

SEQ ID NO: 13 - RNA, 73 nt, Artificial Sequence
  TMirtTert8
guaagaaccuaagguuagugauucaagagaucacuaaccuuagguucuucucaguuuuuucucuuucuuucag

SEQ ID NO: 14 - RNA, 73 nt, Artificial Sequence
  TMirtDKC8
guaaauagcugcagauaguuauucaagagauaacuaucugcagcuauuucucaguuuuuucucuuucuuucag

SEQ ID NO: 15 - RNA, 73 nt, Artificial Sequence
  TMirtDKC9
guaggagaggacuaucuaugguucaagagaccauagauaguccucucuucucaguuuuuucucuuucuuucag

SEQ ID NO: 16 - DNA, 5 bp, Artificial Sequence
  synthetic sequence
ctcag

SEQ ID NO: 17 - RNA, 5 nt, Artificial Sequence
  synthetic sequence
cucag

SEQ ID NO: 18 - RNA, 68 nt, Artificial Sequence
  synthetic sequence
nnnnnnnnnnnnnnnnnnnnnnnnnnnnnnnnnnnnnnnnnnnnnnnnnnnnnnncucagnnnnnnnn

SEQ ID NO: 19 - RNA, 63 nt, Artificial Sequence
  Synthetic sequence
nnnnnnnnnnnnnnnnnnnnnnnnnnnnnnnnnnnnnnnnnncucagnnnnnnnnnnnnnnnn

SEQ ID NO: 20 - RNA, 76 nt, Artificial Sequence
  Synthetic sequence
nnnnnnnnnnnnnnnnnnnnnnnnnnnnnnnnnnnnnnnnnnnnnnnnnnnnnnnnnnnnnnnyunaynnnnnnnn

SEQ ID NO: 21 - RNA, 63 nt, Artificial Sequence
  Synthetic sequence
nnnnnnnnnnnnnnnnnnnnnnnnnnnnnnnnnnnnyunaynnnnnnnnnnnnnnnnnnnnnn

SEQ ID NO: 22 - DNA, 21 bp, Artificial Sequence
  Synthetic sequence
nnnnnnnnnnnnnnnnnnntt

SEQ ID NO: 23 - DNA, 21 bp, Artificial Sequence
  Synthetic sequence
nnnnnnnnnnnnnnnnnnntt

SEQ ID NO: 24 - DNA, 82 bp, Artificial Sequence
  VEGF T1 Primer
tcgatgcagattatgcggatcaaacctgtagacacacccacccacccacatacatacatttatgcggatcaaacctcaccaa

SEQ ID NO: 25 - DNA, 82 bp, Artificial Sequence
  Synthetic sequence
ggccttggtgaggtttgatccgcataaatgtatgtatgtgggtgggtgggtgtgtctacaggtttgatccgcataatctgca

SEQ ID NO: 26 - DNA, 72 bp, Artificial Sequence
  VEGF T2 Primer
tcgagccttgccttgctgctctacctccacatgatcttttttttgtcccactaatgttattggtgtcttcac

SEQ ID NO: 27 - DNA, 72 bp, Artificial Sequence
  Synthetic sequence
ggccgtgaagacaccaataacattagtgggacaaaaaaaagatcatgtggaggtagagcagcaaggcaaggc

SEQ ID NO: 28 - DNA, 21 bp, Artificial Sequence
  Synthetic sequence
ggtgtttcgtcctttccacaa

SEQ ID NO: 29 - DNA, 18 bp, Artificial Sequence
  Synthetic sequence
ggctgcaggtcgacggat

SEQ ID NO: 30 - DNA, 20 bp, Artificial Sequence
  Synthetic sequence
aaggtgaaggtcggagtcaa

SEQ ID NO: 31 - DNA, 20 bp, Artificial Sequence
  Synthetic sequence
gaagatggtgatgggatttc

SEQ ID NO: 32 - DNA, 21 bp, Artificial Sequence
  Synthetic sequence
cggatcaaacctcaccaaggc

SEQ ID NO: 33 - DNA, 19 bp, Artificial Sequence
  Synthetic sequence
agggaggctccttcctcct

SEQ ID NO: 34 - RNA, 78 nt, Artificial Sequence
  Synthetic sequence
gunnnnnnnnnnnnnnnnnnnnnnnnnnnnnnnnnnnnnnnnnnnnnnnnnnnnncucagcucucucucucucucuag

SEQ ID NO: 35 - RNA, 85 nt, Artificial Sequence
  Natural TMirt 877
guagaggagauggcgcaggggacacgggcaaagacuugggguuccugggacccucagacguguguccucuucucccuccucccga

SEQ ID NO: 36 - DNA, 131 bp, Artificial Sequence
  TMirt877v1
gtagaggagatggcgcaggggacacgggcaaagacttgggggttcctgggaccctcagacgtgtgtcctcttctccctcctcccttaattttattatcaccatgacattgactctttcataatctacttag

SEQ ID NO: 37 - DNA, 102 bp, Artificial Sequence
  TMirt877v2
gtagaggagatggcgcagggttcaagagaccctgcgccatctcctttctcagattttattttattagcagcatgagattgactctttcataatctacttaag

SEQ ID NO: 38 - DNA, 74 bp, Artificial Sequence
  MirtFL19
gtgagagaagcgcacacaggcaagcatcattaagccagagcttgtctcagcttgcctgtttgctcttcttttag

SEQ ID NO: 39 - DNA, 119 bp, Artificial Sequence
  Vegfa1-miRNA
gtgagctcgagccctgccgggcgtaaatgtatgtatgtgggtgtttgtgtagcacccacatacatacatttatgctccagcagggcacgcaaagctcagttttttctctttctttcaag

SEQ ID NO: 40 - DNA, 120 bp, Artificial Sequence
  Vegfa8-miRNA
gtgagctcgagccctgccggggctggtagattatgcggattaaatttgtgtagtttgatccgcataatctgtcagctccagcagggcacgcaaagctcagttttttctctttctttcaag

SEQ ID NO: 41 - DNA, 71 bp, Artificial Sequence
  Synthetic sequence
gtrrrnnnnnnnnnnnnnnnntcaagagnnnnnnnnnnnnnnnnnnctcagttttttctctttctttcaag

SEQ ID NO: 42 - DNA, 71 bp, Artificial Sequence
  Synthetic sequence
gtrrrnnnnnnnnnnnnnnnttcaagagannnnnnnnnnnnnnnnnctcagttttttctctttctttcaag

SEQ ID NO: 43 - DNA, 70 bp, Artificial Sequence
  Mirtron 1
gtgagtgccggtgcctgccctggggccgtccctgcgcaggggccgggtgctcaccgcatctgccccgcag

SEQ ID NO: 44 - DNA, 82 bp, Artificial Sequence
  MIrtron 2
gtgggttggggcgggctctggggatttggtctcacagcccggatcccagcccacttaccttggttactctccttccttctag

SEQ ID NO: 45 - DNA, 83 bp, Artificial Sequence
  Mirtron 3
gtgagcaggcgaggctgggctgaacccgtgggtgaggagtgcagcccagctgaggcctctgctgtcttatctgtctcctacag

SEQ ID NO: 46 - DNA, 73 bp, Artificial Sequence
  Mirtron 4
gtgagtggggctcccgggacggcgcccgccctggccctggcccggcgacgtctcacggtccctctcccctcag

SEQ ID NO: 47 - DNA, 76 bp, Artificial Sequence
  MIrtron5
gtgagtgggagccggtggggctggagtaagggcacgcccggggctgccccacctgctgaccaccctccccccacag

SEQ ID NO: 48 - DNA, 80 bp, Artificial Sequence
  Mirtron 6
gtgagtgtggatttggcggggttcgggggttccgacggcgacctcggcgacccctcactcaccgcttcctctttccgcag

SEQ ID NO: 49 - DNA, 104 bp, Artificial Sequence
  Mirtron 7
gtaagcaggggctctgggtgatgtgaggagcaacaggcaccctcctccacagcccctgctcattcctgctgccccccaccccctcccactgcctcaccctctag

SEQ ID NO: 50 - DNA, 77 bp, Artificial Sequence
  MIrtron 8
gtgcggaacgctggccggggcgggaggggaagggacgcccggccggaacgccgcactcacgccgcctctgcccacag

SEQ ID NO: 51 - DNA, 104 bp, Artificial Sequence
  Mrtron 9
gtgaggaggggctggcagggacccctccaagttggggacggcagccagcccctgctcacccctcgccttccttgtctcctctgcccaccttgtcctcacactag

SEQ ID NO: 52 - DNA, 80 bp, Artificial Sequence
  MIrtron 10
gtgagtagtggcgcgcggcggctcggagtacctctgccgccgcgcgcatcggctcagcatgcccgtctctgtctcctcag

SEQ ID NO: 53 - DNA, 68 bp, Artificial Sequence
  Mirtron 11
gtgagtcagggtggggctggccccctgcttcgtgcccatccgcgctctgactctctgcccacctgcag

SEQ ID NO: 54 - DNA, 89 bp, Artificial Sequence
  MIrtron 12
gtgcggacgcggcccagtcctgcccaaggaccggggagcgggaggcccactggggtccctgggctgagcccgtccccacccctccccag

SEQ ID NO: 55 - DNA, 81 bp, Artificial Sequence
  Mirtron 13
gtactaggggcctggaggtttgggctccaagagaagcttgacagagcccacgcccgacccctacttctgtttcttcctaag

SEQ ID NO: 56 - DNA, 79 bp, Artificial Sequence
  Mirtron 14
gtcagtgtgtgggtgtcgggggtgggggcatggcggacctgcgacctgacccctgacacgggcctcctttccactgcag

SEQ ID NO: 57 - DNA, 75 bp, Artificial Sequence
  MIrtron 15
gtgagcagggctggggtacctcccccaacctaggacaccccacttgccctgctgaccccagctgtcccggtgtag

SEQ ID NO: 58 - DNA, 79 bp, Artificial Sequence
  Mirtron 16
gtgagtggggcacggaggctgctgctcccctcggccaccgtgagcagggagccgccctcacaccccctcctctccacag

SEQ ID NO: 59 - DNA, 78 bp, Artificial Sequence
  MIrtron 17
gtcagtgggaccagacaaggggagtggtggttgtctctgcctgggaaactgaccatctttgtttttgtaattcttcag

SEQ ID NO: 60 - DNA, 74 bp, Artificial Sequence
  TMIrtFL25
gttagacaggtcgtacttgtcttcaagagagacaagtacgacctgtctactcagttttttctctttctttcaag

SEQ ID NO: 61 - DNA, 74 bp, Artificial Sequence
  TMirtFL26
gtgaacagcatgggcatttaattcaagagattaaatgcccatgctgttcatcagttttttctctttctttcaag

SEQ ID NO: 62 - DNA, 74 bp, Artificial Sequence
  TMirtVegfa2
gtgggtgggtgtgtctacaggttcaagagacctgtagacacacccacccctcagttttttctctttctttcaag

SEQ ID NO: 63 - DNA, 74 bp, Artificial Sequence
  TMirtVegfa3
gtgaagacaccaataacattattcaagagataatgttattggtgtcttcctcagttttttctctttctttcaag

SEQ ID NO: 64 - DNA, 73 bp, Artificial Sequence
  TMirtVegfa6
gtgggcaaaaaaaagatcatttcaagagaatgatcttttttttgtcccctcagttttttctctttctttcaag

(19) United States
(12) Patent Application Publication
(10) Pub. No.: US 2017/0002350 A1
(43) Pub. Date: Jan. 5, 2017

(54) TAILED MIRTRON EFFECTORS FOR RNAI-MEDIATED GENE SILENCING

(71) Applicant: AGENCY FOR SCIENCE, TECHNOLOGY AND RESEARCH, Singapore (SG)

(72) Inventor: Yiqi SEOW, Singapore (SG)

(21) Appl. No.: 15/100,630

(22) PCT Filed: Oct. 17, 2014
(86) PCT No.: PCT/SG2014/000490

§ 371 Date: May 31, 2016

Related U.S. Application Data
(60) Provisional application No. 61/911,208, filed on Dec. 3, 2013.

(51) Int. Cl. C12N 15/113

(52) U.S. Cl. CPC C12N 15/113; C12N 15/1136; C12N 2310/14; C12N 2310/531; C12N 2320/30; C12N 2310/52

(57) ABSTRACT

The present invention relates to an isolated mirtron capable of binding a target nucleic acid, wherein the isolated mirtron comprises: a) a stem loop structure comprising a 5′ splice site, b) a tail sequence comprising a 3′ splice site and a polypyrimidine-comprising sequence, and c) a branch point sequence. Further, the present invention relates to vectors and pharmaceutical compositions comprising such mirtrons as well as methods of treating a disease comprising administering such pharmaceutical compositions.

### CROSS-REFERENCE TO RELATED APPLICATIONS

This application claims the benefit of priority of U.S. provisional application No. 61/911,208, filed Dec. 3, 2013, the contents of it being hereby incorporated by reference in its entirety for all purposes.

### TECHNICAL FIELD

The present invention generally relates to biochemistry. In particular, the invention covers the use of RNA interfering (RNAi) nucleic acid molecules for gene therapy.

### BACKGROUND

The therapeutic potential of RNA interference (RNAi) pathway, in which short double stranded RNA mediate translational repression or degradation of targeted mRNAs, has been explored extensively with exogenous mimics such as small interfering RNAs (siRNAs), short-hairpin RNAs (shRNAs) or artificial miRNAs (amiRNAs). Recently, mirtrons, which are natural pre-microRNA (pre-miRNA) hairpins produced as introns from mRNA splicing, have been shown to be useful for gene silencing especially when co-delivery of therapeutic genes is required. Unlike promoter-less siRNAs and RNA-polymerase III driven shRNAs, mirtrons can be controlled with an endogenous RNA polymerase II promoter, offering spatiotemporal control. However, canonical mirtrons suffer from sequence constraints due to splicing requirements that limit targetable sequences. A subclass of mirtrons with a 3′ tail that are particular remarkable because most of the sequences required for splicing are located outside of the hairpin (FIG. 1). For natural 3′ tailed mirtrons (TMirts), the precise biogenesis pathway from spliced intron to a hairpin substrate for exportin-5 and DICER has not been elucidated, but has previously been shown to require further nucleolytic processing in flies. As the processing steps and sequence or structural features of TMirts are not well understood, principles behind designing artificial TMirts are not trivial to decipher.

Thus it is an object of the present invention to provide alternative molecules and providing alternative solutions for such interfering nucleic acid molecules.

### SUMMARY

In a first aspect, there is provided an isolated nucleic acid molecule capable of binding a target nucleic acid sequence, wherein the isolated nucleic acid molecule comprises:
a. a stem loop structure comprising
   i. a stem comprising a 5′ arm and a 3′ arm complementarily bound to each other, wherein the 5′ arm and the 3′ arm are connected to each other by a single stranded loop structure;
   ii. a 5′ splice site;
   iii. a nucleic acid sequence that is at least 50% complementary to a nucleotide sequence of the target nucleic acid sequence;
b. a tail sequence comprising
   i. a 3′ splice site;
   ii. a polypyrimidine-comprising sequence; and
c. a branch point sequence comprising a branch point, wherein the branch point sequence is located at the 3′ end of the 3′ arm of the stem loop structure, or up to 4 nucleotides upstream from the 3′ end of the 3′ arm of the stem loop structure, or up to 4 nucleotides downstream from the 5′ end of the tail sequence.

In a second aspect, there is provided a DNA molecule encoding an RNA molecule as defined herein.

In a third aspect, there is provided a vector comprising an isolated nucleic acid as defined herein.

In a fourth aspect, there is provided a pharmaceutical composition comprising an isolated nucleic acid as defined herein.

In a fifth aspect, there is provided a method of treating a disease comprising administering a pharmaceutical composition as defined above to a patient in need of gene therapy.

### DETAILED DISCLOSURE OF EMBODIMENTS

Mirtrons are introns that form pre-miRNA hairpins after splicing to produce RNA interference (RNAi) effectors distinct from Drosha-dependent intronic miRNA. Disclosed herein are design principles for 3′ tailed mirtrons and corresponding mirtrons which have minimal sequence constraints as compared to canonical mirtrons. The mirtrons as defined herein may be used for delivering therapeutic RNAi and may overcome toxicity and off-target issues that are currently facing RNAi strategies.

In one example, there is provided an isolated nucleic acid molecule capable of binding a target nucleic acid sequence, wherein the isolated nucleic acid molecule comprises:
a. a stem loop structure comprising
   i. a stem comprising a 5′ arm and a 3′ arm complementarily bound to each other, wherein the 5′ arm and the 3′ arm are connected to each other by a single stranded loop structure;
   ii. a 5′ splice site;
   iii. a nucleic acid sequence that is at least 50% complementary to a nucleotide sequence of the target nucleic acid sequence;
b. a tail sequence comprising
   i. a 3′ splice site;
   ii. a polypyrimidine-comprising sequence; and
c. a branch point sequence comprising a branch point, wherein the branch point sequence is located at the 3′ end of the 3′ arm of the stem loop structure, or up to 4 nucleotides upstream from the 3′ end of the 3′ arm of the stem loop structure, or up to 4 nucleotides downstream from the 5′ end of the tail sequence.

The isolated nucleic acid may be an asymmetric molecule. For example, in FIG. 2, the isolated nucleic acid molecule is asymmetric along plane A, due to the presence of a tail sequence connected to the 3′ end of the 3′ arm of the stem loop structure.

### Splicing Ability

In general, it is necessary that a mirtron is capable of being spliced out. This involves, for example, a 5′ splice site that can be recognized by U1 or U11 snRNA, a branch point that can be recognized by U2 or U12 snRNA, a 3′ splice site consensus sequence and a polypyrimidine tract between the branch point and the 3′ splice site on the 3′ arm of the stem loop. U1 and U2 snRNAs are used for 5′ splice site and branch point recognition in the canonical splicing pathway (>99% of all splicing events) while U11 and U12 perform the same roles in the minor splicing pathway (<1% of all splicing events). The recognition sites (5′ and 3′ splice sites and branch point) can be different for each pathway, whilst this application generally refers to the major (canonical) pathway, the same concepts are also applicable to the minor splicing pathway.

### 5′ Splice Site Sequence

5′ splice site sequences are either known in the art or can be readily determined. A 5′ splice site of a mirtron as defined herein may comprise the sequence GU as the terminal nucleotides in the 5′ to 3′ direction. In one example, the GU sequence is followed by 3 purines, although splicing may occur if one or two of the purines are substituted with pyrimidines.

The 5′ splice site may be located at the 5′ end of the 5′ arm of the stem loop structure. Alternatively, the 5′ splice site may be located close to the 5′ end of the 5′ arm. For example, the 5′ splice site may be located up 1, 2, 3, 4 or up to 5 nucleotides away from the 5′ end of the 5′ arm that is complementary to the 3′ arm. In one example, the 5′ splice site of the stem loop structure is located at the 5′ end of the stem loop structure.

### 3′ Splice Site Sequence

3′ splice site sequences are either known in the art or can be readily determined. In one example, the 3′ splice site ends with an AG. Therefore, a mirtron as defined herein may comprise a 3′ splice site sequence in which the terminal nucleotides are AG in the 5′ to 3′ direction.

In one example, the 3′ splice site is located at the 3′ end of the tail sequence.

### Branch Point Sequence

A typical human consensus branch point sequence is YUNAY, where Y is C or U and N is any nucleotide. In one example, a branch point sequence, YUNAY, is used wherein N is A, T, C, G or U. The branch point sequence need not conform to this consensus and may be selected from sequences of endogenous branch points identified in any eukaryotic intron, and it is typically but not always found at least 10 nucleotides away from the 3′ splice site. In one example, the branch point sequence is located within 4 nucleotides of the terminal 3′ nucleotide of the hairpin loop. An illustration of these requirements is shown below:

Two examples of the designs when transcribed into RNA are shown as below in which N refers to any of the ribonucleotide with the bases Adenine (A), Cytosine (C), Guanine (G), or Uracil (U). N may also refer to the deoxyribonucleotides with the bases adenine (A), cytosine (C), Guanine, or Thymidine (T) in the DNA encoding the RNA sequence.

```
SEQ ID NO: 18
                                                  NN
5′-NNNNNNNNNNNNNNNNNNNNNN N
      ||||||||||||||||||||||  N
NNNNNNNNNNNNNNNNNNNNNNNNN
N NN
CUCAGNNNNNNNN-3′
SEQ ID NO: 19
        NN NN
5′-N NNNNNNNNNNNNNNNNNNN N
      |  |||||||||||||||||||  N
NNNNNNNGACUCNNNNNNNNNNNNN
N NN
NNNNNNNN-3′
```

Branch point sequence (CUCAG) in bold and underlined, where A is the branch point. Terminal 3′ nucleotide (N) in bold

Other examples different from the aforementioned would be:

```
SEQ ID NO: 20
                                                  NN
5′-NNNNNNNNNNNNNNNNNNNNNN N
      ||||||||||||||||||||||  N
NNNNNNNNNNNNNNNNNNNNNNNNN
N NN
NNNNNNNNYUNAYNNNNNNNN-3′
SEQ ID NO: 21
        NN NN
5′-N NNNNNNNNNNNNNNNNNNN N
      |  |||||||||||||||||||  N
NNNNNNNNNNNNNYANUYNNNNNNN
N NN
NNNNNNNN-3′
```

Branch point sequence (CUCAG) in bold and underlined, where A is the branch point. Terminal 3′ nucleotide (N) in bold.

Thus, in one example, the branch point sequence may consist of 3, 4, 5, 6, 7 or up to 8 nucleotides. The branch point sequence further comprises a branch point. The branch point may be an adenosine. Alternatively, the branch point may be a guanosine. For example, in case the branch point sequence is 8 nucleotides long, the branch point can be in any position, such as position 1, 2, 3, 4, 5, 6, 7 or 8.

In one example, the branch point sequence is CTCAG (SEQ ID NO: 16)(DNA), where A is the branch point. In another example, the branch point sequence is CUCAG (SEQ ID NO: 17)(RNA), where A is the branch point.

### Polypyrimidine Tract

The polypyrimidine tract is a region of messenger RNA (mRNA) that promotes the assembly of the spliceosome, the protein complex specialized for carrying out RNA splicing during the process of post-transcriptional modification.

In one example, the 3′ polypyrimidine tract leading into the 3′ splice site can be 15 or greater than 15 nucleotides long. The mirtron as disclosed herein may therefore comprise a 3′ polypyrimidine tract of 15 or more nucleotides in length, for example 18 or more, 20 or more, or 25 or more, 30 or more, 35 or more, 40 or more, 45 or more, 50 or more, 55 or more, 60 or more, 65 or more nucleotides in length. In one example, the polypyrimidine tract comprises 15 nucleotides or more. For example, the polypyrimidine tract may be 17, 18, 19, 20, 21, 22, 23, 24, 25, 26, 27, 28, 29, 30 nucleotides long.

“Polypyrimidine tract” means that a series of nucleotides are present in the RNA sequence substantially consisting of pyrimidines, i.e. cytosine (C) and uracil (U). In one example, the tract does not consist entirely of pyrimidines. In one example, it comprises of at least 70%, 80%, 90% or greater than 95% pyrimidines. In one example, the polypyrimidine tract comprises of at least 70% of pyrimidines.

The polypyrimidine tract is located between the 3′ splice site and the branch-point and located 1-50 nucleotides upstream of the 3′ splice site. For example, it may be located 1-10, 10-20, 20-30, 30-40 or 40-50 nucleotides upstream of the 3′ splice site.

### Stem Loop Structure

In the endogenous RNAi pathway, DICER recognises pre-miRNA structures characterised by a dsRNA stem of around 21-23 nts and a loop at one end resulting in a stem-loop structure. Following the actions of RNA exosome complex to degrade the 3′ tail and lariat debranching enzyme to debranch the intron lariat, a spliced mirtron may therefore form a stem-loop structure via regions within 5′ and 3′ arms that are base complementary and be processed by DICER.

The DICER is fairly flexible in its substrate specificity and the stem-loop structure formed from the mirtrons as defined herein have little limits other than being of the correct length. As long as the stem-loop structure is processed by DICER then the dsRNA will be shuttled into RISC for silencing purposes.

To be recognised by DICER, the length of the dsRNA duplex that is required for RNAi and the length of the stem-loop structure that is to be used has some significance. It has been found that siRNAs designed with a 19 nt region of base pairing and 2 nt overhangs at each 3′ end (see below) are the most effective length of siRNAs in mammalian cells, although longer siRNAs and blunt ended siRNAs have been designed and shown to be effective.

| (SEQ ID NO: 22) | |
|---|---|
| 3′-TTNNNNNNNNNNNNNNNNNNN -5′ | Sense |
| (SEQ ID NO: 23) | |
| 5′- NNNNNNNNNNNNNNNNNNNTT-3′ | Anti-sense |

Endogenous miRNAs also share a preference for 2 nt 3′ tails following processing by DROSHA′ and DICER whilst variation in the length is seen with the detection of anti-sense strands showing sizes ranging from 21-23 nts (inclusive of overhang).

Analysis of some mammalian mirtrons suggests that unlike miRNAs and siRNAs there appears to be a preference of single-nucleotide overhangs at the 5′ and 3′ ends of the stem-loop structure, although a 2 nt overhang is seen at the 3′ end of the 5′ arm species following processing by DICER. However the length of the anti-sense species produced falls often into the same 21-23 nt group as seen with miRNAs.

Thus, the 5′ and the 3′ arm of the stem of the mirtrons as defined herein may each comprise 29 nucleotides or less. For example, the 5′ arm may comprise of about 5-10 nucleotides, about 8-12 nucleotides, about 10-15 nucleotides, about 12-17 nucleotides, about 15-20 nucleotides, about 17-23 nucleotides, about 20-25 nucleotides, about 22-27 nucleotides, about 25-29 nucleotides. The 3′ arm may comprise 5-10 nucleotides, about 8-12 nucleotides, about 10-15 nucleotides, about 12-17 nucleotides, about 15-20 nucleotides, about 17-23 nucleotides, about 20-25 nucleotides, about 22-27 nucleotides, about 25-29 nucleotides. The short stem loop of 29 nucleotides or less is believed to prevent processing by the Drosha enzyme and allows the mirtron as disclosed herein to bypass the Drosha processing pathway.

The mirtrons as defined herein are sufficiently base-complementary in the 5′ and 3′ region in order for a stem loop structure to form.

The 5′ and the 3′ arm of the stem may be complementarily bound to each other. For example, the 5′ arm and the 3′ arm may be 100% complementarily bound to one another. In one example, the 5′ arm and the 3′ arm may be about 95%, about 90%, about 85%, about 80% or about 75% complementarily bound to one another. The complementary binding between the 2 arms is due to the complementary base-pairing (RNA) between A and U; G and C; or G and U. Alternatively, the complementary binding between the 2 arms is due to the complementary base-pairing (DNA) between A and T, G and C; or G and T.

Alternatively, the 5′ and 3′ arm may comprise of up to about 5%, up to about 10%, up to about 15%, up to about 20%, up to about 25% nucleotides mismatch.

The single stranded loop structure connecting the 5′ arm and 3′ arm of the stem may be 10 nucleotides or less. Alternatively, the single stranded loop structure may be 9, 8, 7, 6, 5, 4, 3, or 2 nucleotides long.

The stem loop structure may also comprise 1 or 2 nucleotides of 5′ overhang.

### Target Sequence

The selection of siRNA against a gene of interest starts with an annotated target mRNA sequence including its 5′ and 3′ un-translated regions and its splice, polymorphic and allelic variants. Because the coding sequence is the most reliable mRNA sequence information available; it is commonly targeted. The UTRs are generally less well characterised but can also be targeted with, similar gene knockdown efficiency. In one example, targeting sequences that contain known binding sites for mRNA-binding proteins such the exon-exon junction complex are avoided. Additional considerations can be made in identifying siRNAs that target orthologs in more than one species or all splice variants of a gene.

The mirtrons as defined herein may comprise a nucleic acid sequence that is at least partially complementary to a sequence in the target mRNA sequence. It may be designed to have complete complementarity to the target mRNA sequence to effect elimination of the mRNA transcript. Alternatively, the mirtrons as defined herein may be designed with partial complementarity to the target mRNA in order to direct translational repression. A partially complementary nucleic acid sequence may be partially complementary to the target mRNA in relation to the entire length of the nucleic acid sequence. Alternatively, partially complementary may be limited to a 2-9 nucleotide seed, region. The “seed region” refers to a region at position 2-9 of the nucleic acid that is often disproportionally involved in target binding. Partial complementarity may be at least 40%, 50%, 60%, 70%, 80%, 90%, at least 95% or at least 98% complementary. In one example, the nucleic acid sequence is at least 50% complementary to a nucleotide sequence of the target nucleic acid sequence, i.e. the target mRNA sequence.

The nucleic acid sequence that is at least partially complementary to a sequence in the target nucleic acid sequence may be located on the 5′ or 3′ arm of the mirtron as disclosed herein. The nucleic acid may be located on the guide strand of the stem loop. As used herein, the “guide strand” refers to the strand that is incorporated into the RNA-induced silencing complex (RISC) during processing of the RNA. The “passenger strand” is the complementary strand that is degraded during processing. In one example, the 5′ arm of the stem-loop structure is the guide strand while the 3′ arm is the passenger strand. In another example, the 5′ arm of the stem-loop structure is the passenger strand while the 3′ arm is the guide strand. In one example, there is provided an isolated nucleic acid molecule as defined herein, wherein the 5′ or the 3′ arm of the stem-loop structure is a guide strand.

In one example, the target mRNA sequence may be selected from a group consisting of Renilla luciferase, dystrophia myotonica protein kinase (DMPK), Firefly luciferase, Vascular endothelial growth factor A (VEGFA), Telomerase RNA component (TerC), Telomerase reverse transcriptase 1 (Tert1) and Dyskerin (DKC1). The mirtron as disclosed herein may be selected from a group consisting of TMirt877v3.1 (SEQ ID NO: 1), TMir877v3.2 (SEQ ID NO: 2), DMPK TMirt5 (SEQ ID NO: 3), DMPKTMirt13 (SEQ ID NO: 4), TMirtFL19 (SEQ ID NO: 5), TMirtVEGF1 (SEQ ID NO: 6), TMirtVEGF4 (SEQ ID NO: 7), TMirtVEGF7 SEQ ID NO: 8), TMirtVEGF8 (SEQ ID NO: 9), TmirtTerC2 (SEQ ID NO: 10), TmirtTerC4 (SEQ ID NO: 11), TmirtTert7 SEQ ID NO: 12), TmirtTert8 (SEQ ID NO: 13), TmirtDKC8 (SEQ ID NO: 14) and TMirtDKC9 (SEQ ID NO: 15).

### Polynucleotides

The mirtrons as defined herein comprise a nucleic acid sequence that has the ability to enter into the RNAi pathway as a miRNA bypassing Drosha. The mirtrons as defined herein may be delivered on its own, i.e. without further nucleotide sequence. However, in one example, the mirtron as disclosed herein may be delivered in a polynucleotide construct. In one example, the nucleic acid as disclosed herein may take the place of a natural intron within the sequence of a gene in the construct. The disclosure also relates to polynucleotide constructs comprising nucleic acid sequences which express a mirtron as disclosed herein. The constructs may comprise one or more nucleic acid sequences that encode one or more polypeptides.

In one example, the mirtron as defined herein may be an RNA or a DNA. In one example, there is provided a DNA molecule encoding an RNA molecule as defined herein. A DNA molecule is said to “encode” an RNA molecule when it can be transcribed into RNA in its native state or when manipulated by methods well known to those skilled in the art. The DNA molecule encoding the RNA molecule as defined herein may comprise a coding sequence, or a non-coding sequence or a combination of both. Similarly, the RNA molecule may also comprise a coding sequence or a non-coding sequence, or a combination of both. The “coding sequence” of a DNA or RNA molecule refers to a DNA or RNA sequence that encodes for a protein. The “non-coding” sequence of a DNA or RNA molecule refers to a DNA or RNA sequence that does not encode for a protein.

The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the disclosure may be provided in isolated or purified form.

A nucleic acid sequence such as a DNA or RNA which “encodes” a polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the disclosure, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

In one example, a polynucleotide of the disclosure comprises a sequence corresponding to any of the mammalian mirtron sequences as defined herein or a variant of one of these specific sequences. For example, a variant may be a substitution, deletion or addition variant of any of these nucleic acid sequences. A variant may comprise 1, 2, 3, 4, 5, up to 10, up to 20, up to 30, up to 40, up to 50, up to 75 or more nucleic acid substitutions and/or deletions from the sequences provided. If the designed sequence is being provided in place of a natural intron within a gene in a construct, the sequence must be capable of being spliced. The sequence must be capable of entering the RNAi pathway and targeting the desired sequence for downregulation.

Suitable variants may be at least 70% homologous to polynucleotide sequence defined herein, preferably at least 80 or 90% and more preferably at least 95%, 97% or 99% homologous thereto. Methods of measuring homology are well known in the art and it will be understood by those of skill in the art that in the present context, homology is calculated on the basis of nucleic acid identity. Such homology may exist over a region of at least 15, preferably at least 30, for instance at least 40, 60, 100, 200 or more contiguous nucleotides. Such homology may exist over the entire length of the polynucleotide sequence.

Methods of measuring polynucleotide homology or identity are known in the art. For example, the UWGCG Package provides the BESTFIT program which can be used to calculate homology (e.g. used on its default settings). The PILEUP and BLAST algorithms can also be used to calculate homology or line up sequences:

### Vectors

The mirtrons as defined herein may be provided in the form of an expression cassette which includes control sequences operably linked to the inserted sequence, thus allowing for expression of the polynucleotide containing the mirtrons as defined herein in vivo in a targeted subject species. These expression cassettes, in turn, are typically provided within vectors (e.g., plasmids or recombinant viral vectors). Such an expression cassette may be administered directly to a host subject. Alternatively, a vector comprising a polynucleotide of the disclosure may be administered to a host subject. Preferably the polynucleotide is prepared and/or administered using a genetic vector. A suitable vector may be any vector which is capable of carrying a sufficient amount of genetic information, and allowing expression of the polynucleotide of the disclosure.

The present disclosure thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the disclosure. Other suitable vectors would be apparent to persons skilled in the art.

Thus, a polynucleotide of the disclosure may be provided by delivering such a vector to a cell and allowing transcription from the vector to occur. Preferably, a polynucleotide of the disclosure or for use in the disclosure in a vector is operably linked to a control sequence which is capable of providing for the expression of the coding sequence by the host cell, i.e. the vector is an expression vector.

“Operably linked” refers to an arrangement of elements wherein the components so described are configured so as to perform their usual function. Thus, a given regulatory sequence, such as a promoter, operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence.

A number of expression systems have been described in the art, each of which typically consists of a vector containing a gene or nucleotide sequence of interest operably linked to expression control sequences. These control sequences include transcriptional promoter sequences and transcriptional start and termination sequences. The vectors of the disclosure may be for example, plasmid, virus or phage vectors provided with an origin of replication, optionally a promoter for the expression of the said polynucleotide and optionally a regulator of the promoter. A “plasmid” is a vector in the form of an extrachromosomal genetic element. The vectors may contain one or more selectable marker genes, for example an ampicillin resistance gene in the case of bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example to allow in vivo expression of the polynucleotide.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

Promoters and other expression regulation signals may be selected to be compatible with the host cell for which expression is designed. For example, mammalian promoters, such as β-actin promoters, may be used. Tissue-specific promoters are especially preferred. Mammalian promoters include the metallothionein promoter which can be induced in response to heavy metals such as cadmium.

In one example a viral promoter is used to drive expression from the polynucleotide. Typical viral promoters for mammalian cell expression include the SV40 large T antigen promoter, adenovirus promoters, the Moloney murine leukaemia virus long terminal repeat (MMLV LTR), the mouse mammary tumor virus LTR promoter, the rous sarcoma virus (RSV) LTR promoter, the SV40 early promoter, the human cytomegalovirus (CMV) IE promoter, adenovirus, including the adenovirus major late promoter (Ad MLP), HSV promoters (such as the HSV IE promoters), or HPV promoters, particularly the HPV upstream regulatory region (URR). All these promoters are readily available in the art.

In one example, the promoter is a Cytomegalovirus (CMV) promoter. A preferred promoter element is the CMV immediate early (IE) promoter devoid of intron A, but including exon 1. Thus the expression from the polynucleotide may be under the control of hCMV IE early promoter. Expression vectors using the hCMV immediate early promoter include for example, pWRG7128, and pBC12/CMV and pJW4303. A hCMV immediate early promoter sequence can be obtained using known methods. A native hCMV immediate early promoter can be isolated directly from a sample of the virus, using standard techniques. U.S. Pat. No. 5,385,839, for example, describes the cloning of a hCMV promoter region. The sequence of a hCMV immediate early promoter is available at Genbank #M60321 (hCMV Towne strain) and X17403 (hCMV Ad169 strain). A native sequence could therefore be isolated by PCR using PCR primers based on the known sequence. A suitable hCMV promoter sequence could also be isolated from an existing plasmid vector. Promoter sequences can also be produced synthetically.

A polynucleotide or vector of the disclosure may comprise an untranslated leader sequence. In general the untranslated leader sequence has a length of from about 10 to about 200 nucleotides, for example from about 15 to 150 nucleotides, preferably 15 to about 130 nucleotides. Leader sequences comprising, for example, 15, 50, 75 or 100 nucleotides may be used. Generally a functional untranslated leader sequence is one which is able to provide a translational start site for expression of a coding sequence in operable linkage with the leader sequence.

Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as well as a bovine growth hormone terminator sequence.

Expression systems often include transcriptional modulator elements, referred to as “enhancers”. Enhancers are broadly defined as a cis-acting agent, which′ when operably linked to a promoter/gene sequence, will increase transcription of that gene sequence. Enhancers can function from positions that are much further away from a sequence of interest than other expression control elements (e.g. promoters), and may operate when positioned in either orientation relative to the sequence of interest. Enhancers have been identified from a number of viral sources, including polyoma virus, BK virus, cytomegalovirus (CMV), adenovirus, simian virus 40 (SV40), Moloney sarcoma virus, bovine papilloma virus and Rous sarcoma virus. Examples of suitable enhancers include the SV40 early gene enhancer, the enhancer/promoter derived from the long terminal repeat (LTR) of the Rous Sarcoma Virus, and elements derived from human or murine CMV, for example, elements included in the CMV intron A sequence.

A polynucleotide or vector according to the present disclosure may additionally comprise a signal peptide sequence. The signal peptide sequence is generally inserted in operable linkage with the promoter such that the signal peptide is expressed and facilitates secretion of a polypeptide encoded by coding sequence also in operable linkage with the promoter.

Typically a signal peptide sequence encodes a peptide of 10 to 30 amino acids for example 15 to 20 amino acids. Often the amino acids are predominantly hydrophobic. In a typical situation, a signal peptide targets a growing polypeptide chain bearing the signal peptide to the endoplasmic reticulum of the expressing cell. The signal peptide is cleaved off in the endoplasmic reticulum, allowing for secretion of the polypeptide via the Golgi apparatus.

The nucleic acid molecule can be introduced directly into the recipient subject, such as by standard intramuscular or intradermal injection; transdermal particle delivery; inhalation; topically, or by oral, intranasal or mucosal modes of administration. The molecule alternatively can be introduced ex vivo into cells which have been removed from a subject. In this latter case, cells containing the nucleic acid molecule of interest are re-introduced into the subject.

Each of these delivery techniques requires efficient expression of the nucleic acid in the transfected cell, to provide a sufficient amount of the therapeutic product. Several factors are known to affect the levels of expression obtained, including transfection efficiency, and the efficiency with which the gene or sequence of interest is transcribed and the mRNA translated.

The vector of the present disclosure may be administered directly as “a naked nucleic acid construct”, preferably further comprising flanking sequences homologous to the host cell genome. As used herein, the term “naked DNA” refers to a vector such as a plasmid comprising a polynucleotide of the present disclosure together with a short promoter region to control its production. It is called “naked” DNA because the vectors are not carried in any delivery vehicle. When such a vector enters a host cell, such as a eukaryotic cell, the proteins it encodes are transcribed and translated within the cell.

The vector of the disclosure may thus be a plasmid vector, that is, an autonomously replicating, extrachromosomal circular or linear DNA molecule. The plasmid may include additional elements, such as an origin of replication, or selector genes. Such elements are known in the art and can be included using standard techniques. Numerous suitable expression plasmids are known in the art. For example, one suitable plasmid is pSG2. This plasmid was originally isolated from Streptomyces ghanaensis. The length of 13.8 kb, single restriction sites for HindIII, EcoRV and PvuII and the possibility of deleting non-essential regions of the plasmid make pSG2 a suitable basic replicon for vector development.

Alternatively, the vectors of the present disclosure may be introduced into suitable host cells using a variety of viral techniques which are known in the art, such as for example infection with recombinant viral vectors such as retroviruses, herpes simplex viruses and adenoviruses.

In one example, the vector itself may be a recombinant viral vector. Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors. In the case of viral vectors, administration of the polynucleotide is mediated by viral infection of a target cell.

A number of viral based systems have been developed for transfecting mammalian cells.

For example, a selected recombinant nucleic acid molecule can be inserted into a vector and packaged as retroviral particles using techniques known in the art. The recombinant virus can then be isolated and delivered to cells of the subject either in vivo or ex vivo. Retroviral vectors may be based upon the Moloney murine leukaemia virus (Mo-MLV). In a retroviral vector, one or more of the viral genes (gag, pol & env) are generally replaced with the gene of interest.

A number of adenovirus vectors are known. Adenovirus subgroup C serotypes 2 and 5 are commonly used as vectors. The wild type adenovirus genome is approximately 35 kb of which up to 30 kb can be replaced with foreign DNA.

There are four early transcriptional units (E1, E2, E3 & E4), which have regulatory functions, & a late transcript, which codes for structural proteins. Adenovirus vectors may have the E1 and/or E3 gene inactivated. The missing gene(s) may then be supplied in trans either by a helper virus, plasmid or integrated into a helper cell genome. Adenovirus vectors may use an E2a temperature sensitive mutant or an E4 deletion. Minimal adenovirus vectors may contain only the inverted terminal repeats (ITRs) & a packaging sequence around the transgene, all the necessary viral genes being provided in trans by a helper virus. Suitable adenoviral vectors thus include Ad5 vectors and simian adenovirus vectors.

Viral vectors may also be derived from the pox family of viruses, including vaccinia viruses and avian poxvirus such as fowlpox vaccines. For example, modified vaccinia virus Ankara (MVA) is a strain of vaccinia virus which does not replicate in most cell types, including normal human tissues. A recombinant MVA vector may therefore be used to deliver the polypeptide of the disclosure.

Addition types of virus such as adeno-associated virus (AAV) and herpes simplex virus (HSV) may also be used to develop suitable vector systems.

As an alternative to viral vectors, liposomal preparations can alternatively be used to deliver the nucleic acid molecules of the disclosure. Useful liposomal preparations include cationic (positively charged), anionic (negatively charged) and neutral preparations, with cationic liposomes particularly preferred. Cationic liposomes may mediate intracellular delivery of plasmid DNA and mRNA.

As another alternative to viral vector systems, the nucleic acid molecules of the present disclosure may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). Other particulate systems and polymers can also be used, for example, polymers such as polylysine, polyarginine, polyornithine, spermine, spermidine, as well as conjugates of these molecules.

In one example, the vector may be a targeted vector, that is a vector whose ability to infect or transfect or transduce a cell or to be expressed in a host and/or target cell is restricted to certain cell types within the host subject, usually cells having a common or similar phenotype.

In one example, there is provided a vector comprising an isolated nucleic acid as defined herein. The vector may comprise more than one isolated nucleic acids, for example 1, 2, 3 or more isolated nucleic acids.

### Pharmaceutical Compositions

Formulation of a composition comprising a molecule of the disclosure, such as a polynucleotide, or vector as described above, can be carried out using standard pharmaceutical formulation chemistries and methodologies all of which are readily available to the reasonably skilled artisan. For example, compositions containing one or more molecules of the disclosure can be combined with one or more pharmaceutically acceptable excipients, or vehicles. Auxiliary substances, such as wetting or emulsifying agents, pH buffering substances and the like, may be present in the excipient, or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like.

Such compositions may be prepared, packaged, or sold in a form suitable for bolus administration or for continuous administration. Injectable compositions may be prepared, packaged, or sold in unit dosage form, such as in ampoules or in multi-dose containers containing a preservative. Compositions include, but are not limited to, suspensions, solutions, emulsions in oily or aqueous vehicles, pastes, and implantable sustained-release or biodegradable formulations. Such compositions may further comprise one or more additional ingredients including, but not limited to, suspending, stabilizing, or dispersing agents. In one example of a composition for parenteral administration, the active ingredient is provided in dry (for e.g., a powder or granules) form for reconstitution with a suitable vehicle (e.g., sterile pyrogen-free water) prior to parenteral administration of the reconstituted composition. The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Other parentally-administrable compositions which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules. Anionic and neutral liposomes are widely available and well known for delivering nucleic acid molecules. Cationic lipid preparations are also well known vehicles for use in delivery of nucleic acid molecules. Suitable lipid preparations include DOTMA (N-[1-(2,3-dioleyloxy)propyl]-N,N,N-trimethylammonium chloride), available under the tradename Lipofectin™, and DOTAP (1,2-bis(oleyloxy)-3-(trimethylammonio)propane). These cationic lipids may preferably be used in association with a neutral lipid, for example DOPE (dioleyl phosphatidylethanolamine). Still further transfection-facilitating compositions that can be added to the above lipid or liposome preparations include spermine derivatives and membrane-permeabilizing compounds such as GALA, Gramicidine S and cationic bile salts.

Alternatively, the nucleic acid molecules of the present disclosure may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). Other particulate systems and polymers can also be used, for example, polymers such as polylysine, polyarginine, polyornithine, spermine, spermidine, as well as conjugates of these molecules.

The formulated compositions will include an amount of the molecule (e.g. vector) of interest which is sufficient to mount an immunological response. An appropriate effective amount can be readily determined by one of skill in the art. Such an amount will fall in a relatively broad range that can be determined through routine trials. The compositions may contain from about 0.1% to about 99.9% of the vector and can be administered directly to the subject or, alternatively, delivered ex vivo, to cells derived from the subject, using methods known to those skilled in the art.

### Delivery Methods

Once formulated the compositions can be delivered to a subject in vivo using a variety of known routes and techniques. For example, a composition can be provided as an injectable solution, suspension or emulsion and administered via parenteral, subcutaneous, epidermal, intradermal, intramuscular, intraarterial, intraperitoneal, intravenous injection using a conventional needle and syringe, or using a liquid jet injection system. Compositions can also be administered topically to skin or mucosal tissue, such as nasally, intratracheally, intestinal, rectally or vaginally, or provided as a finely divided spray suitable for respiratory or pulmonary administration. Other modes of administration include oral administration, suppositories, and active or passive transdermal delivery techniques. Particularly in relation to the present disclosure, compositions may be administered directly to the gastrointestinal tract. As explained above, the mirtron as defined herein may be delivered as a construct within a suitable vector by any gene delivery system available for gene therapy, such as virus vectors, and plasmids in liposome formulation. Alternatively, the compositions can be administered ex vivo, for example delivery and reimplantation of transformed cells into a subject are known (e.g., dextran mediated transfection, calcium phosphate precipitation, electroporation, and direct microinjection into nuclei).

### Delivery Regimes

The compositions are administered to a subject in an amount that is compatible with the dosage formulation and that will be prophylactically and/or therapeutically effective. An appropriate effective amount will fall in a relatively broad range but can be readily determined by one of skill in the art by routine trials.

As used herein, the term “prophylactically or therapeutically effective dose” means a dose in an amount sufficient to alleviate, reduce, cure or at least partially arrest symptoms and/or complications from a disease.

Prophylaxis or therapy can be accomplished by a single direct administration at a single time point or by multiple administrations, optionally at multiple time points. Administration can also be delivered to a single or to multiple sites. Those skilled in the art can adjust the dosage and concentration to suit the particular route of delivery. In one example, a single dose is administered on a single occasion. In an alternative example, a number of doses are administered to a subject on the same occasion but, for example, at different sites. In a further example, multiple doses are administered on multiple occasions. Such multiple doses may be administered in batches, i.e. with multiple administrations at different sites on the same occasion, or may be administered individually, with one administration on each of multiple occasions (optionally at multiple sites). Any combination of such administration regimes may be used.

Different administrations may be performed on the same occasion, on the same day, one, two, three, four, five or six days apart, one, two, three, four or more weeks apart. Preferably, administrations are 1 to 5 weeks apart, more preferably 2 to 4 weeks apart, such as 2 weeks, 3 weeks or 4 weeks apart. The schedule and timing of such multiple administrations can be optimised for a particular composition or compositions by one of skill in the art by routine trials.

Dosages for administration will depend upon a number of factors including the nature of the composition, the route of administration and the schedule and timing of the administration regime. The dose will also vary according to the severity of the condition, age, and weight of the patient to be treated. A physician will be able to determine the required route of administration and dosage for any particular patient. Optimum dosages may vary depending on the relative potency of the nucleic acids, and can generally be estimated based on EC₅₀s found to be effective in vitro and in in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the nucleic acid, the age, weight and condition of the subject to be treated, the severity of the disease and the frequency and route of administration.

### Therapeutic Treatment and Strategies

The mirtrons as defined herein are for use in modifying expression of a gene in a mammalian cell. They may be used as research tools or have a therapeutic purpose. The mirtrons as defined herein may be used to treat any disease which can be treated by gene silencing or knock down. As used herein, the term “treat” or “treatment” includes any and all uses which remedy a disease state or symptoms, prevent the establishment of disease, or otherwise prevent, hinder, retard, or reverse the progression of disease or other undesirable symptoms in any way whatsoever. Hence, “treatment” includes prophylactic and therapeutic treatment.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (eg. sheep, cows, horses, donkeys, pigs), laboratory test animals (eg. rabbits, mice, rats, guinea pigs, hamsters), companion animals (eg. cats, dogs) and captive wild animals (eg. foxes, deer, dingoes). The terms do not denote a particular age. Thus, both adult and newborn individuals are intended to be covered. The subject will preferably be a human, but may also be a domestic livestock, laboratory subject or pet animal.

The disclosure provides for a method of treating a disease comprising administering a pharmaceutical composition as defined herein to a patient in need of gene therapy. The term “administering” and variations of that term including “administer” and “administration”, includes contacting, applying, delivering or providing a compound or composition of the invention to an organism, or a surface by any appropriate means. In one example, there is provided a use of a pharmaceutical composition as defined herein for the manufacture of a medicament for treating a patient in need of gene therapy. In another example, there is provided a pharmaceutical composition as defined herein for use in treating a patient in need of gene therapy.

The pharmaceutical composition may be used in the treatment of a genetic disease by reducing or eliminating the expression of a target defective gene. In one example, the target gene may comprise one or more dominant gain-of-function mutation(s).

The disease to be treated may be selected from a group consisting of Dentatorubropallidoluysian atrophy, Huntington's disease, Spinobulbar muscular atrophy or Kennedy disease, Spinocerebellar ataxia Type 1, Spinocerebellar Type 2, Spinocerebellar ataxia Type 3 or Machado-Joseph disease, Spinocerebellar ataxia Type 6, Spinocerebellar ataxia Type 7, Spinocerebellar ataxia Type 17, Fragile X syndrome, Fragile XE mental retardation, Friedreich's ataxia, Myotonic dystrophy, Spinocerebellar ataxia Type 8, Spinocerebellar Type 12, Marfan Syndrome, myoeproliferative disorders, ALS, Parkinson's disease, angiogenesis and cancer.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1. Proposed biogenesis of (a) canonical mirtrons and (b) tailed mirtrons. Canonical mirtrons have strong sequence constraints due to need for spliceosome recognition while tailed mirtrons have less. For example, a canonical mirtron has 5′ splice site constraints. It has to incorporate a polypyrimidine tract on the 3′ arm, with a corresponding purine rich region on the 5′ arm. Furthermore, a canonical mirtron has a large loop to incorporate the branch point. 3′ tailed mirtrons also have 5′ splice site constraints. However, it does not have constraints relating to incorporation of the polypyrimidine tract, since the polypyrimidine tract is in the tail region. A 3′ tailed mirtron is able to have a smaller loop, thus making it a more effective Dicer substrate. The sequence of events following splicing is still not currently clear, thus the interplay between 3′ degradation and debranching can have important consequences on mirtron biogenesis. Both canonical and 3′ tailed mirtrons in the form of pre-miRNA are brought to the spliceosome where they undergo the process of splicing. After splicing, both RNA of canonical or 3′ tailed mirtrons may either be debranched or optionally undergo the process of 3′ degradation prior to debranching. If the RNA is not debranched, the hairpin that can halt 3′ RNA degradation by RNA exosome is not able to form either. In the case of a canonical mirtron, the process of debranching without prior 3′ degradation leads to the formation of a Dicer substrate for effective targeted knockdown. In the case of a 3′ tailed mirtron, the process of 3′ degradation followed by debranching leads to the formation of a Dicer substrate that can be further processed for effective knockdown.

FIG. 2. The diagram shows a 3′-tailed mirtron comprising a stem loop structure and a 3′ tail. The stem loop structure has a 5′ and a 3′ arm connected by a loop, and comprises the 5′ splice site on the 5′ arm. The tail comprises the polypyrimidine tract and the 3′ splice site. The branch point sequence may be located at the 3′ end of the 3′ arm of the stem loop structure. It can also be located up to 4 nucleotides upstream from the 3′ end of the 3′ arm of the stem loop structure, or up to 4 nucleotides downstream from the 5′ end of the tail sequence as shown here in the diagram.

FIG. 3. Development of effective 3′-tailed mirtrons. HEK cells were transfected with 100 ng of psiCheck2.2 targets with target sequences in the 3′UTR of Renilla luciferase and 500 ng of mirtron plasmid and assayed 2 days later. a-c, Fluorescence quantification, Renilla luciferase knockdown and design of different TMirt design with has-mir-877 and mmu-mir-1224 guide strands in a pEGFP-Mirt vector. d, High throughput sequencing was performed with 18-25 nt RNA from HEK293 cells transfected with TMirt877v3.2 and v3.3. Sequences that were aligned to mirtron sequences were collected, 3′ tail removed and aligned to chart the frequency of individual nucleotides appearing in the small RNA species harvested from the cells. Red nucleotides denote guide and passenger strands. e, TMirts based on canonical mirtrons designed against DMPK were compared to the parent mirtrons based on splicing efficiency (eGFP fluorescence) and Renilla luciferase knockdown f, High throughput sequencing of small RNAs from HEK293 cells transfected with 500 ng of the relevant plasmids. * p<0.05, ** p<0.01, *** p<0.001 vs NAD control, n=3 biological replicates for each experiment. Error bars reflect standard deviation. g, Different versions of TMirt design with has-mir-877 guide strands are shown (natural Mirt877 (SEQ ID NO:35), version 1 (TMirt877v1, SEQ ID NO: 36), version 2 (TMirt877v2, SEQ ID NO: 37), version 3.1 (TMirt877v3.1, SEQ ID NO. 1) and version 3.2 (TMirtv3.2, SEQ ID NO: 2). Guide strands are marked by lighter coloured text.

FIG. 4. 3′-tailed mirtrons effect strong knockdown when targeted to the coding region. a, Sequence of TMirtFL19 (SEQ ID NO:5) compared to MirtFL19 (SEQ ID NO:38). Mutations made are indicated by substitutions of the nucleotides within the boxes. b, HEK293 cells were transfected with 100 ng of psiCheck2.2 and 0, 250 ng or 800 ng of peGFP-MirtFL19, peGFP-TMirtFL19 or Song of a linear U6p-FL19 shRNA PCR product and topped up to 900 ng of nucleic acids with peGFP-NAD. Cells were harvested 2 days after transfection. c, HEK293 cells were transfected with 100 ng of psiCheck2.2, and 500 ng of mirtrons/NAD. Cells were harvested 2 days after transfection. d, HEK293 cells were transfected with 100 ng of psiCheck2.2, 500 ng of mirtrons/NAD, and 500 ng of peGFP-C1, peGFP-XPO5 or pAdvantage. Cells were harvested 2 days after transfection. e, HEK293 cells were transfected with 100 ng of psiCheck2.2, and 500 ng of mirtrons/NAD. Cells were harvested 2 days after transfection. * p<0.05, ** p<0.01, *** p<0.001 vs NAD control, n=3 biological replicates for each experiment unless otherwise stated. Error bars reflect standard deviation.

FIG. 5. 3′ Tailed mirtrons against VEGFA results in functional knockdown of VEGF-A in a cell culture model of hypoxia. a, HEK293 cells were transfected with 500 ng of NAD/TMirts and 100 ng of the matched psiCheck2.2 targets (T1 and T2 contains concatamers of the target sequences in the 3′UTR). Cells were harvested 2 days after transfection. b, HEK293 cells were transfected with 500 ng of mirtrons/NAD and subjected to hypoxic conditions 1 day after transfection for hours before harvest. qRT-PCR of VEGF was performed RNA harvested from the cells normalized against GAPDH. ELISA was performed on the cell culture supernatant and total human VEGF secreted in 500 μl of medium as measured by ELISA is indicated. c-e, Sequence of TMirts (TmirtVegfal (SEQ ID NO:6) and TMirtVegfa8 (SEQ ID NO:9)) compared to their miRNA equivalents (Vegfa1-miRNA (SEQ ID NO:39) and Vegfa8 miRNA (SEQ ID. NO:40)). Mutations made are indicated by substitutions of the nucleotides within the boxes. d, HEK293 cells were transfected with 500 ng of NAD/TMirts mutants/miRNA mimics and 100 ng of the matched psiCheck2.2 VEGFA T1. Cells were harvested 2 days after, transfection. * p<0.05, ** p<0.01, *** p<0.001 vs NAD control, n=3 biological replicates for each experiment unless otherwise stated. Error bars reflect standard deviation.

FIG. 6. Design features of 3′ Tailed Mirtrons (SEQ ID NOs: 41 and 42). 3′ Tailed mirtrons can be designed to be precise in biogenesis and strong in splicing by incorporating these features. 1) 5′ guide/passenger strand must start with the canonical 5′ splice site; 2) a short hairpin connects the guide and passenger strands; 3) the 2 nucleotide overhang on the 3′ end of the hairpin designed to mimics endogenous miRNAs should end with the branch point A (pink) followed by another nucleotide; 4) the branch point and polypyrimidine should be optimized for high splicing efficiency based on branch point prediction software and a long uninterrupted polypyrimidine tract.

FIG. 7. TMirts were extracted from a human mirtron database (http://ericlailab.com/mammalian_mirtrons/hg19/) (SEQ ID Nos 43-59). The mature strands are identified and branched points are predicted using prediction algorithm. The majority of human TMirts are found to have 3′ ends within 5 nucleotides of the predicted branch point (13/17).

FIG. 8. Artificial mirtrons previously designed against DMPK6 were converted to tailed mirtrons. The sequences of DMPKTmirt5 (SEQ ID NO:3) and 13 (SEQ ID NO:4) are shown here. These were tested for knockdown of their respective luciferase constructs.

FIG. 9. TMirts designed to Firefly luciferase (TMirtFL25 (SEQ ID NO:60) and 26 (SEQ ID NO:61)) with the guide strand in the 5′ arm or 3′ arm of the hairpin are shown here.

FIG. 10. Mirtrons targeting VEGFA were designed and tested for knockdown using luciferase constructs. The sequences of TMirtVegfa2 (SEQ ID NO:62), 3 (SEQ ID NO:63), 4 (SEQ ID NO 7), 6 (SEQ ID No:64) and 7 (SEQ ID NO:8) are shown here.

FIG. 11. TMirts (TMirtTerC2, TmirtTerC4, TMirtTert7, TMirtTert8, TMirtDKC8 and TMirtDKC9) were designed to target the components of the telomerase complex—Tert1, DKC1 and TerC RNA, and shown to lead to knock down of their respective targets.

### EXAMPLES

Non-limiting examples of the invention, including the best mode, and a comparative example will be further described in greater detail by reference to specific Examples, which should not be construed as in any way limiting the scope of the invention.

### Example 1

### General Methods

Unless otherwise stated, all chemicals were obtained from Sigma-Aldrich and all enzymes used were obtained from New England Biolabs.

### Plasmids

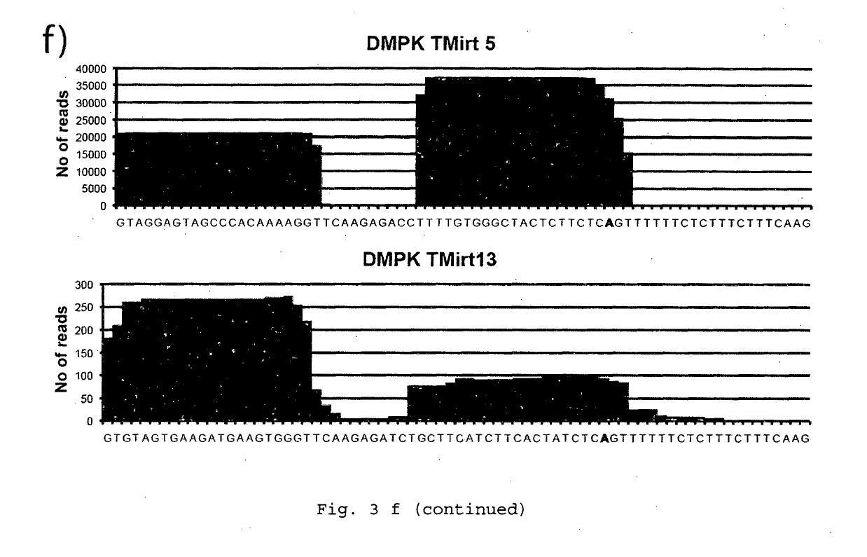

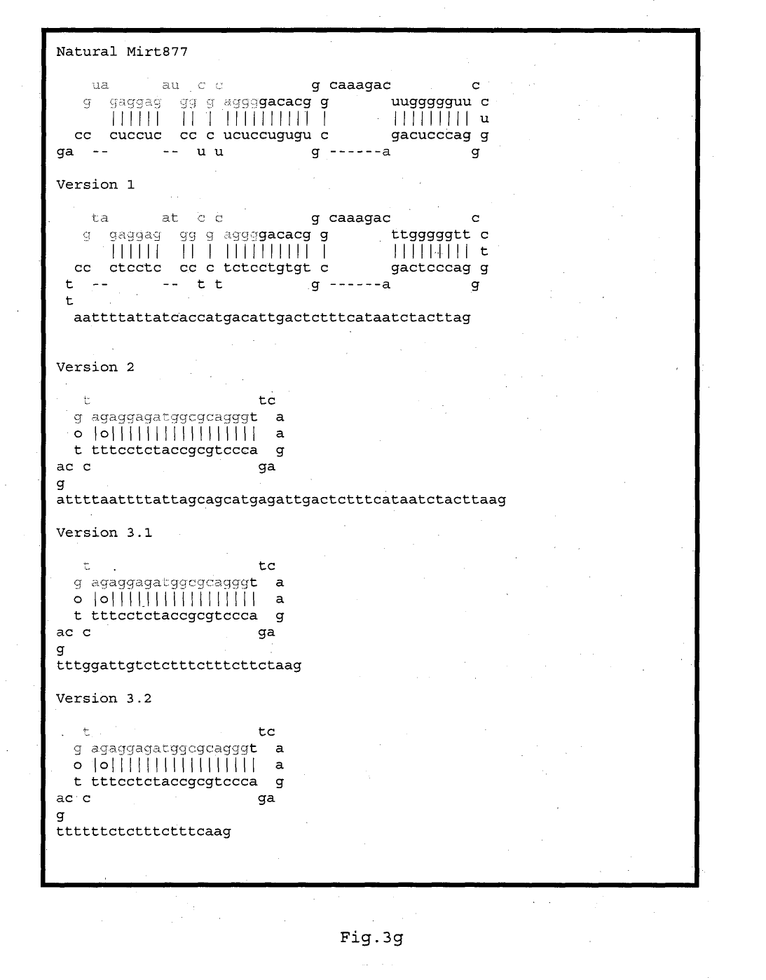

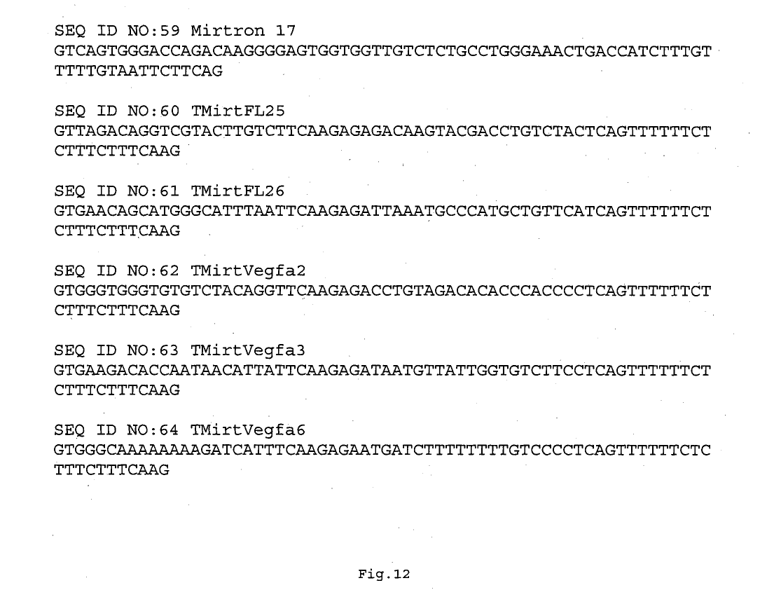

pEGFP-Hirt and cloning of mirtrons was previously described (Sibley et al, 2011). Briefly, a BbsI-excised sequence was placed between two fragments of eGFP and introns introduced in that site with annealed oligonucleotides (FIG. 3b). Luciferase targets for Mirt877, Mirt1224, DMPKMirt5 and DMPKMirt13 were described previously. Other luciferase targets were produced by annealing and ligating the relevant targets as concatamers (Table 1) using oligonucleotides into psiCheck2.2 (FIG. 11) (kind gift from Dr Marc Weinberg) downstream of Renilla luciferase between XhoI and NotI sites.

The relevant shRNAs used for comparison were produced by PCR of the U6 promoter with the reverse complement of the shRNA followed by a reverse U6 primer.

peGFP-XPO5 was described previously and pAdVantage was obtained from Promega.

TABLE 1

| | |
|---|---|
| Primer | Sequence |

pEGFP-Mirt, psiCheck2.2, shRNA and DMPK Cloning

| | |
|---|---|
| Mirtron | 5′-caag - mirtron sequence forward-3′ and |
| cloning | 5′-cgtc - mirtron sequence reverse-3′ |
| VEGFA T1 | 5′- |
| for (Mirt | tcgatgcagattatgcggatcaaacctgtagacacacccacccacccaca |
| 1, 2, 7, | tacatacatttatgcggatcaaacctcaccaa-3′ |
| 8)(SEQ ID | |
| NO: 24) | |
| (SEQ ID NO: | 5′- |
| 25) | ggccttggtgaggtttgatccgcataaatgtatgtatgtgggtgggtggg |
| | tgtgtctacaggtttgatccgcataatctgca-3′ |
| VEGFA T2 | 5′-tcgagccttgccttgctgctctacctccacatgatcttttttttgt |
| for (Mirt | cccactaatgttattggtgtcttcac-3′ |
| 3, 4, 5, 6) | |
| (SEQ ID NO: | |
| 26) | |
| (SEQ ID NO: | 5′-ggccgtgaagacaccaataacattagtgggacaaaaaaaagatcat |
| 27) | gtggaggtagagcagcaaggcaaggc-3′ |
| shRNA | 5′-ggtgtttcgtcctttccacaa - mirtron sequence |
| cloning | reverse-3′ |
| (SEQ ID NO: | |
| 28) | |
| U6F | 5′-ggctgcaggtcgacggat-3′ |
| (SEQ ID NO: | |
| 29) | |

Quantitative PCR

| | |
|---|---|
| hGAPDH F | 5′-aaggtgaaggtcggagtcaa-3′ |
| (SEQ ID NO: | |
| 30) | |
| hGAPDH R | 5′-gaagatggtgatgggatttc-3′ |
| (SEQ ID NO: | |
| 31) | |
| VEGFA F | 5′-cggatcaaacctcaccaaggc-3′ |
| (SEQ ID NO: | |
| 32) | |
| VEGFA R | 5′-agggaggctccttcctcct-3′ |
| (SEQ ID NO: | |
| 33) | |

Cell Culture, Transfection, ELISA and qRT-PCR

HEK293 cells (ATCC) were cultured in DMEM Glutamax supplemented with 10% FBS and antibiotics and incubated at 37° C. in 5% CO2. Transfection of HEK cells were carried out with using Lipofectamine 2000 (Invitrogen) in HEK293 cells as per manufacturer's instruction in 24 well plates. When the experiment required a control NAD plasmid or shRNA, an molar amount corresponding to the mirtron plasmid was used. The cells were imaged and lysed 48 hours post-transfection. To induce hypoxia in HEK293 cells, cells were transfected with 500 ng of mirtron plasmids in 24 well plates. 24 hours after transfection, medium was changed to medium that had been left overnight in the hypoxic chamber (5% CO2, 1% O2, depleted via addition of nitrogen) and cells culture in hypoxia for 24 hours before collection of cell culture medium for human VEGF Quantikine ELISA (R&D Systems) as per manufacturer's instructions and qRT-PCR using Trizol (Life Technologies) extraction, M-MLV reverse transcriptase (Promega) and Maxima SYBR Gold qPCR mix (Thermo Scientific) as per manufacturers' instructions.

### Fluorescence Quantification and Dual Luciferase Assay

The luciferase assay was performed with Promega Dual-Luciferase® Reporter Assay System as per manufacturer's instruction. Briefly, HEK cells were lysed in 100 μl of passive lysis buffer and background-subtracted eGFP fluorescence was measured in a 96 well format with 15 μl of each sample using (Tecan). 15 μl of Luciferase Buffer II was added to the sample and luminescence was measured. 15 μl of Stop & Glo Buffer was then added to measure Renilla luciferase activity. The Renilla luciferase signal is then normalized to the firefly luciferase signal for all luciferase experiments except for experiments in which firefly luciferase knockdown was measured, in which case, the normalization was reversed.

### High Throughput Sequencing

HEK cells were grown in 24-well plates to 80% confluence and transfected with 500 ng of each mirtron plasmid per well. Small RNA libraries were prepared with the “Small RNA v1.5 Sample Prep Kit” following the manufacturer's instructions (Illumina). Briefly, total RNA was isolated from each transfection by Trizol extraction and pooled. The RNA was ligated with 3′ RNA adaptor modified to target small RNAs with 3′ hydroxyl groups, and then with 5′ RNA adaptor. Reverse transcription followed by PCR was performed to select for adapter ligated fragments and double-stranded DNA libraries were size selected by PAGE purification (6% TBE PAGE). Libraries were sequenced on a Genome Analyzer IIx for 36 cycles following manufacturer's protocols. The image analysis and base calling were done using Illumina's GA Pipeline. Adapters were trimmed with Biopieces remove_adapter script and remaining sequences were aligned against full length mirtron hairpins.

### Statistics

All experiments, unless otherwise stated, were all performed in triplicates. All error bars used in this report are standard deviations. Statistical significance was determined by one-tailed student's t-test assuming equal variance.

### Examples of Design Principles for Exemplary Mirtrons

A search can be made in databases which are available that archive experimentally tested siRNA sequences from the literature (http://sirecords.umn.edu/siRecords/ or http://sirna.cgb.ki.se/).

Several siRNA sequence selection algorithms have been developed (http://sirna.cgb.ki.se/ and http://jura.wi.mit.edu/bioc/siRNA). A small number of these algorithms will also consider the secondary structure and accessibility of the targeted mRNA which may affect efficiency (http://www.cs.hku.hk/˜sirna/ and http://sfold.wadsworth.org/index.pl). However, if sequences are to be designed without the aid of a design programme or registered supplier, the following rules are advisable where possible to ensure increased specificity and efficacy, and these are incorporated into design algorithms.

For example, the nucleotides are 21 nt in length with symmetric 2 nt 3′ overhangs (i.e. strand length is 19 nt and at the 3′ end of each strand there is a 2 nt overhang, typically TT). Although longer dsRNAs appear to have the advantage that they can be transfected at lower concentrations than conventional siRNAs without loss of gene silencing, they also appear to be more likely to induce non-specific responses or mediate other effects on cell viability.

The primary sequence is asymmetric (different nucleotides at each end).

The 5′ ends of the anti-sense/guide strand are enriched with A and U nucleotides, e.g. U or A at position 1 of anti-sense/guide strand, A and U richness in positions 1-7 of the anti-sense/guide strand.

The 3′ ends of the anti-sense/guide strand are enriched with G and C nucleotides, e.g. C or G (more commonly C) at position 19 of the anti-sense/guide strand.

The G and C content is about 30-55% (from analysis of functional siRNAs). Too low may destabilise the siRNA duplex and reduce affinity for target mRNA binding. Too high and RISC loading/cleavage may be impaired.

Internal repeats or palindromes which could form secondary intra-strand secondary structures that can interfere with the RNAi process are avoided.

Positions 9-14 e.g. A or U at position 10 of the anti-sense/guide strand are designed to have low stability. The A or U at position 10 is at the cleavage site and is believed to promote catalytic RISC-mediated passenger strand and substrate cleavage.

Extended runs of altering G and C pairs (more than 7) or runs of more than three guanines should be avoided.

siRNA sequences containing putative immuno-stimulatory motifs in either strand are filtered out to minimize toxicities and non-specific silencing effects.

Without being bound by hypothesis, it is believed that the strand with the less stable 5′ end, owing either to weaker base pairing or introduction of mismatches, is favourably loaded into RISC. The above rules agree with this hypothesis of thermodynamic asymmetry and may contribute to the bias for selection of the anti-sense/guide strand in RISC. Chemical methods of preventing passenger-strand use have also been introduced and can be used (e.g. Dharmacon's ON-Target™ siRNA). It is important to design a siRNA with these rules such that the anti-sense/guide strand is incorporated preferentially.

Following the design process each candidate siRNA should be examined for similarity to all other mRNA transcripts that might unintentionally be targeted at a genome-wide level. Each strand of an siRNA duplex, once assembled into RISC, can guide recognition of fully and partially complementary target mRNAs, referred to as ON and OFF targets respectively. Identifying possible OFF-targets can be achieved by entering the complementary siRNA strand sequences into a BLAST search and looking for sequence homology. Particular attention should be paid to positions 2-9, the seed region, which has a major role in siRNA specificity. It is advised that at least 3 mismatches should be made between positions 2 and 19 from OFF-targets and the mismatches near the 5′ and in the centre of the examined strand should be assigned higher significance. In contrast anti-sense/guide strand position 1 and nucleotides at 3′ overhangs have little, if any, contribution to the specificity of target recognition.

Within the endogenous RNAi pathway two actions can be performed depending on the degree of base-pairing between the mRNA target and the anti-sense strand of the active RNAi species. Complete base-pairing directs mRNA cleavage whereas the predominantly encountered partial base-pairing, including nucleotides 2-9 that form the seed region that is disproportionally involved in target binding, directs translational repression.

The first case of complete base-pairing is what is commonly aimed for when RNAi is exploited synthetically as in most cases elimination of the mRNA transcript is desired. However the endogenous miRNAs follow the second scenario of incomplete base-pairing, and as expected, a synthetic construct, be it a siRNA, shRNA or miRNA mimic, can be designed to have this effect. The result is that the transcription of the mRNA is repressed, so although it is still present the protein is not produced and you effectively see gene silencing. Furthermore, in the endogenous pathway miRNAs actually target the 3′ untranslated region of the mRNA transcripts whereas synthetically designed constructs normally target the open-reading frame that is coding. The reason miRNAs target the 3′UTR and not the ORF, appears to be so that the necessary proteins required for repression are not blocked by the ribosomal proteins translating the mRNA.

The mirtrons as defined herein may target the 3′UTR or the ORF of the mRNA. When targeting the 3′ UTR it is necessary to make sure that the seed region that lies at nucleotides 2-9 of the anti-sense strand pairs to the target. When designing constructs 8 nucleotide sequences repeated in the 3′UTR may be used to maximise our chances of success. A suitable programme can be used to identify these repeats together with all acceptable variants that can be accommodated.

### Example 2

### Human 3′ Tailed Mirtron Hairpins are Mostly Defined by 5′ Splice Site and Branch Point

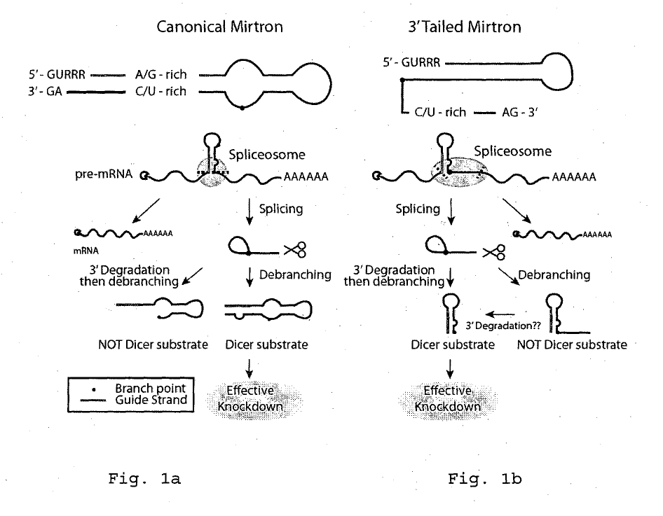

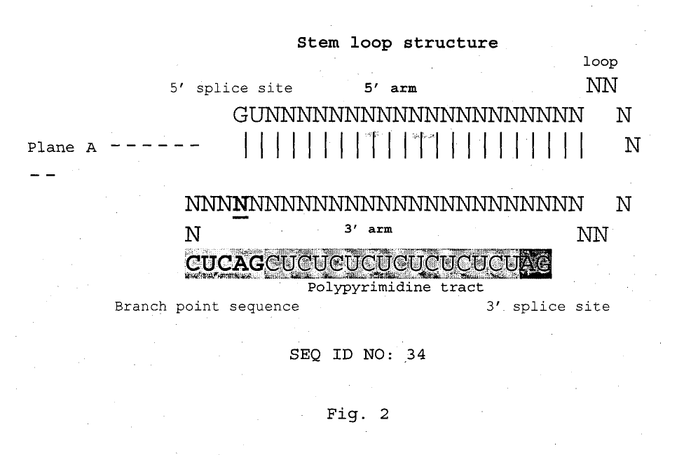

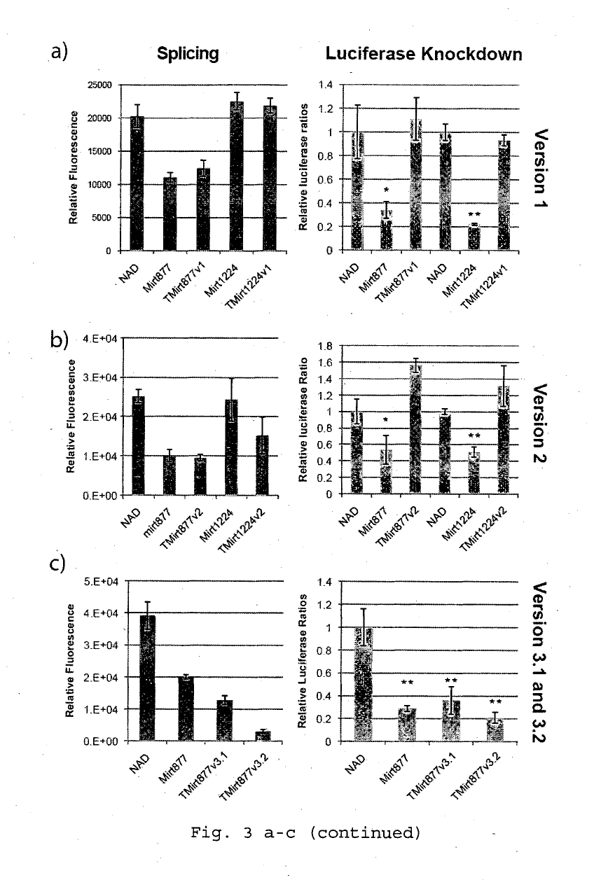

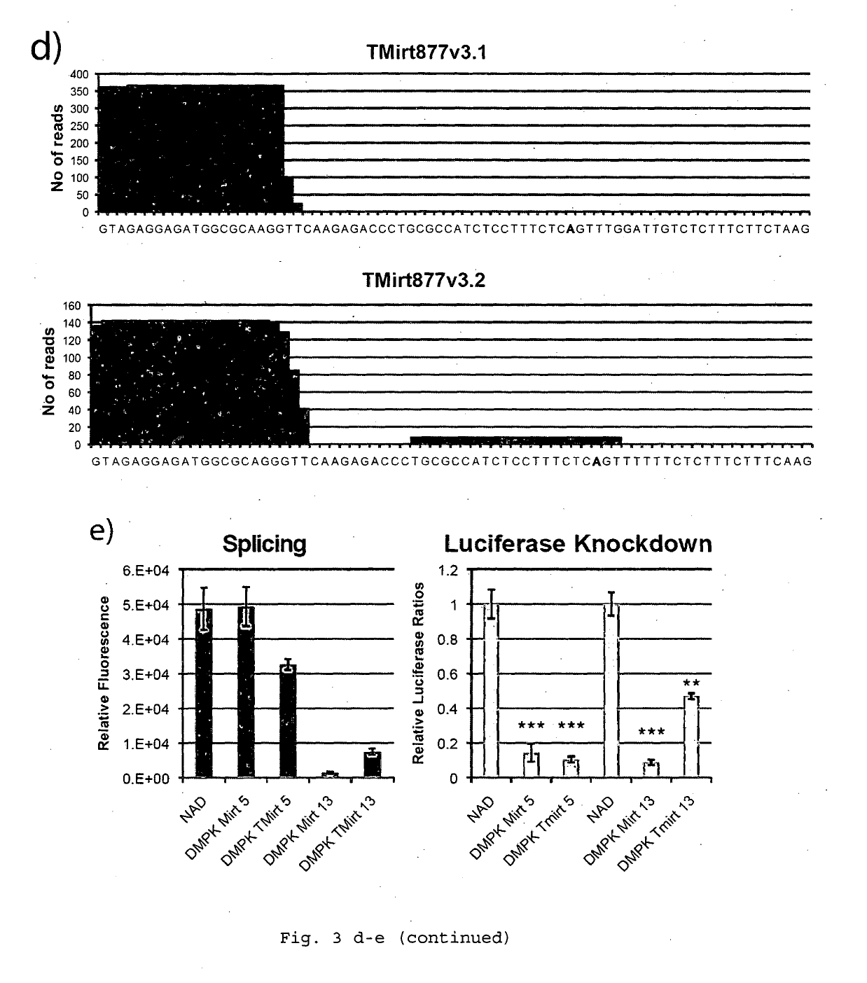

It has been previously suggested that Drosophila mirtrons are debranched before RNA exosome trimming as the RNase components of the RNA exosomes were capable of degrading an in vitro transcribed intron down to the hairpin structure. However, it is equally plausible that the order of the RNA exosome-trimming and debranching steps are interchangeable or concurrent in the cell (FIG. 1). In yeast, RNA lariats accumulate in yeast strain lacking debranching activity, implying that the 3′-5′ exonucleolytic activity of the RNA exosome can only degrade RNA as far as the branch point. If the RNA is not debranched, the hairpin that can halt RNA exosome degradation is not able to form either. Thus if the predicted 3′ end of the hairpin structure is located away from the branch point, exosome trimming prior to or concurrent with debranching may lead to 3′ ends that are either too short or too long and can lead to either inconsistent processing and off-target effects or ineffective production of mature miRNA (FIG. 1). Thus it is likely that TMirts preferentially defined by the branch point by natural selection.

Using a human mirtron database (http://ericlailab.com/mammalian_mirtrons/hg19/), TMirts were extracted, its mature strands identified and branched point predicted using prediction algorithm (FIG. 7). As expected, the majority of human TMirts have 3′ ends within 5 nucleotides of the predicted branch point (13/17).

### Example 3

### Artificial 3′-Tailed Mirtrons can be Designed Against Transcripts of Interest

Given the rules governing natural 3′-tailed mirtrons, an attempt was made to generate artificial TMirts nested within eGFP that could target transcripts of interest. In order not to contend with the potency of the guide strand, initial designs were made based on the mature hsa-miR-877 and mmu-miR-1224—canonical mirtrons with the guide strands in the 5′ arm previously shown to be functional against targets located in the 3′ untranslated region (3′UTR) of Renilla luciferase13. The 3′ splice site of the natural mirtron hairpins was first replaced with a longer polypyrimidine tract and 3′ splice site of intron 6 of human NDUFS1, an intron that had been previously used and is currently being using as a negative control for mirtron experiments (NAD). The splicing would still depend on the predicted branch point located within the hairpin, located at roughly the same distance from the 3′ splice site as the branch point in NDUFS1 intron 6 (FIGS. 3a and 3g v1). Despite comparable splicing with the parent mirtrons based on eGFP fluorescence, no knockdown was detectable with these constructs in HEK293 cells. Unsurprisingly, the lack of knockdown is probably because the branch point is located too far from the 3′ end of the hairpin, thus resulting in the degradation of the hairpin 3′ arm prior to debranching.

Instead of the original hairpin, a minimal hairpin with a highly complementary 3′ arm was used to remove the polypyrimidine tract and branch point within the hairpin. This also has the additional property of freeing up the sequence constraints of the guide strand that plagues development of artificial mirtrons. The branch point of NDUFS1 intron 6 and pyridimine tract was inserted 3′ of the hairpin (v2). Both miR-877 and miR-877-derived tailed mirtrons spliced relatively well but unfortunately, no knockdown was observed with this design (FIGS. 3b and 3g).

As the polypyrimidine tract of NDUFS1 intron 6 is rather long and branch point ill-defined, ‘ideal’ polypyrimidine tracts of different length was substituted in without potential adenosines to act as branch points. The third iterations of tailed mirtrons (v3.1 and v3.2) with the miR-877 guide strand were spliced efficiently and resulted in knockdown of the luciferase target comparable to the parental mir-877 (FIGS. 3c and 3g). High throughput sequencing of small RNAs produced in HEK293 cells transfected with these constructs demonstrated that the guide strand produced by the tailed mirtron is as designed with a very precisely defined 5′ end (FIG. 3d). As the v3.1 resulted in stronger knockdown, subsequent mirtrons were designed with v3.1 sequence.

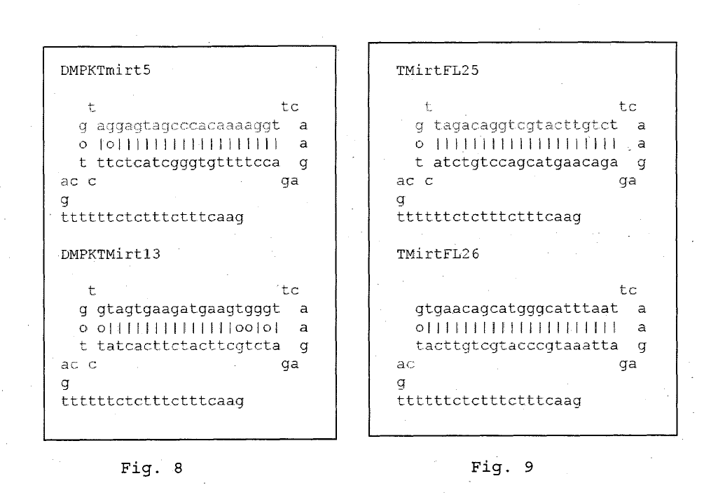

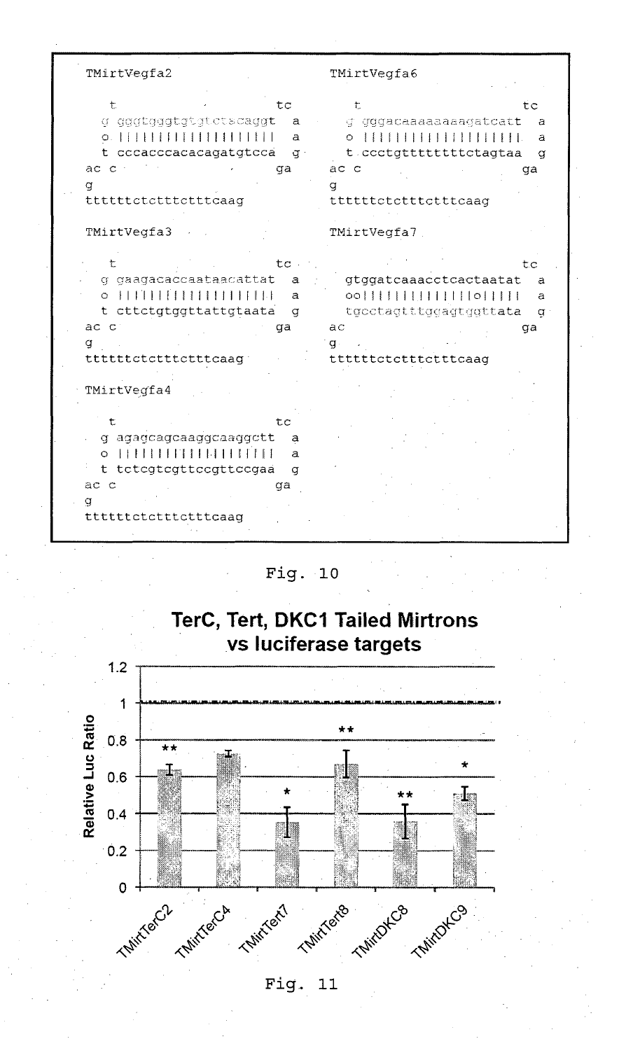

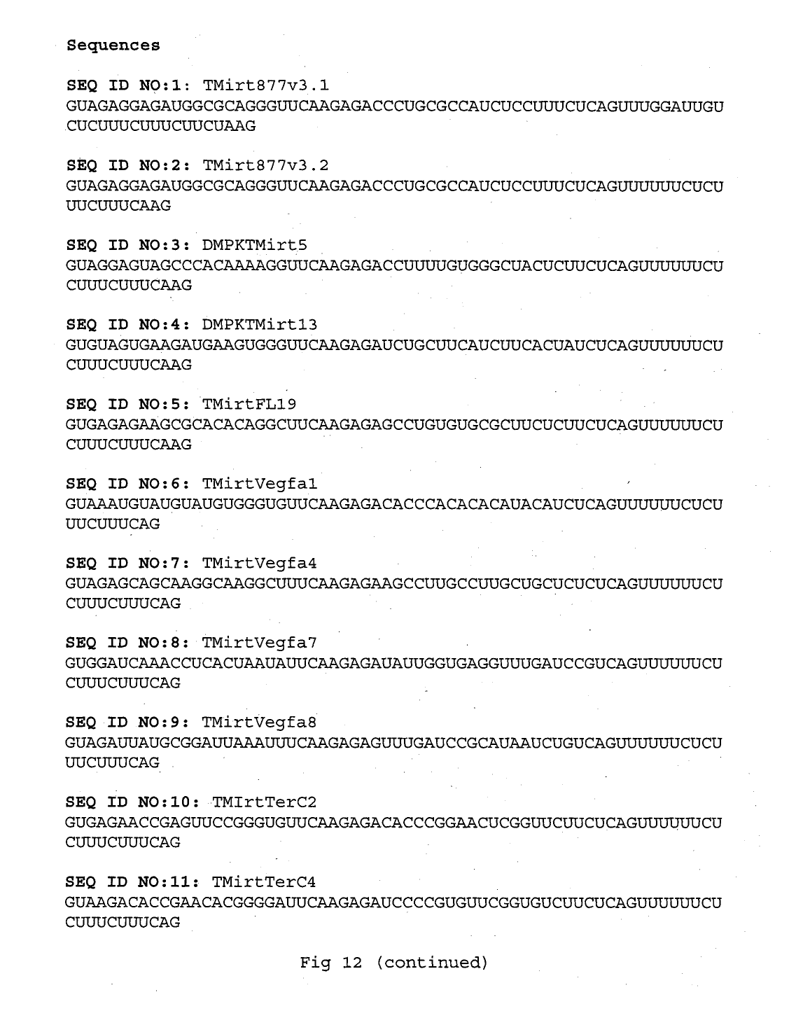

Next, to demonstrate that this design was generally applicable, artificial mirtrons previously designed against DMPK were converted to tailed mirtrons (FIG. 8) and were similarly able to induce knockdown of their respective luciferase constructs at levels comparable to their parent mirtron (FIG. 3e). More importantly, sequencing of the small RNAs produced also validates the design and demonstrates precision of processing (FIG. 3f).

### Example 4

### 3′ Tailed Mirtron are can Effect Knockdown when Targeted to Coding Regions

Interestingly, the relative counts of mature short RNAs derived from high throughput sequencing were much higher with the tailed versions of the DMPK mirtrons compared to the canonical designs previously described, despite the same preparation protocol being used. The relatively low levels of mature species being generated for canonical mirtrons, which have so far be undetectable with Northern blots, probably results in the difficulty in translating mirtrons which demonstrate strong knockdown with targets in the 3′UTR of luciferase to knockdown of an endogenous target's protein coding region (unpublished results). Although the sequencing experiments were performed independently thus not directly comparable and splicing levels were comparable based on eGFP levels (FIG. 3e), the large changes could arise from how the lariat structure is processed (FIG. 1), with competition between debranching and exonuclease degradation. Thus, compared to artificial canonical mirtrons, artificial TMirts can allow greater flexibility of sequences and higher levels of mature species resulting in better knockdown.

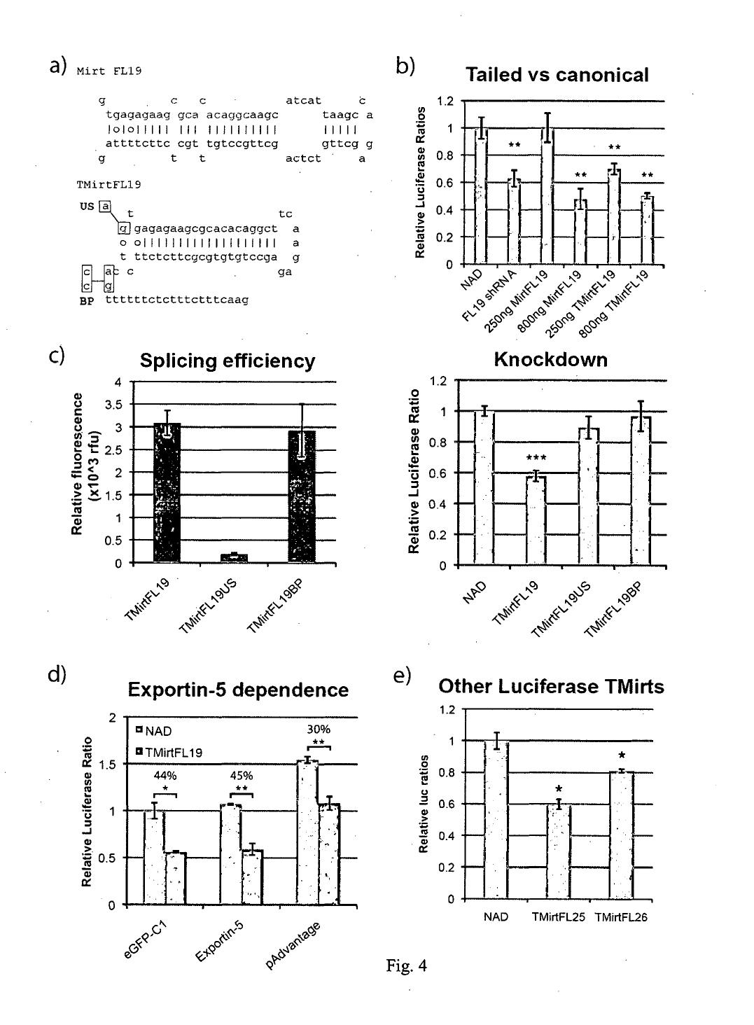

Mirtrons targeted to Firefly luciferase coding sequence using the mirtron design algorithm were previously found to be unable to result in strong knockdown in spite of the guide strand having strong knockdown potential as demonstrated by the corresponding shRNAs. In particular, FL19 with a shRNA design demonstrated strong knockdown but a mediocre knockdown as a mirtron. Converting FL19 to a TMirt design resulted in knockdown of Firefly luciferase that was dose-dependent that was notably stronger at lower concentrations of mirtrons (FIGS. 4a and b). This demonstrates that TMirts can target coding regions of genes, not just targets in the 3′UTR.

### Example 5

TMirt-Mediated Knockdown is Dependent on Splicing and not Exportin-5

Mutation of the guanine of the 5′ splice site of TMirtFL19 to adenosine (US) abrogated splicing based on eGFP fluorescence and knockdown (FIG. 4c). Mutation of the branch point AG to pyrimidines CC (BP) elongates the polypyrimidine tract and shifts the branch point further up the hairpin, resulting in strong splicing but absolutely no knockdown. These mutants demonstrate that knockdown is dependent on precise splicing to generate the pre-miRNA hairpin.

Co-expression of exportin-5, which is responsible export of most pre-miRNA, did not increase the knockdown of the luciferase target (FIG. 4d). Addition of pAdvantage, which expresses VA-1 RNA, an exportin-5 substrate which competes with miRNAs but also stabilizes mRNAs, decreased the relative knockdown but as it also altered the relative ratio of Firefly to Renilla luciferase, the decrease is not as dramatic as expected if export of TMirtFL19 was dependent on exportin-5.

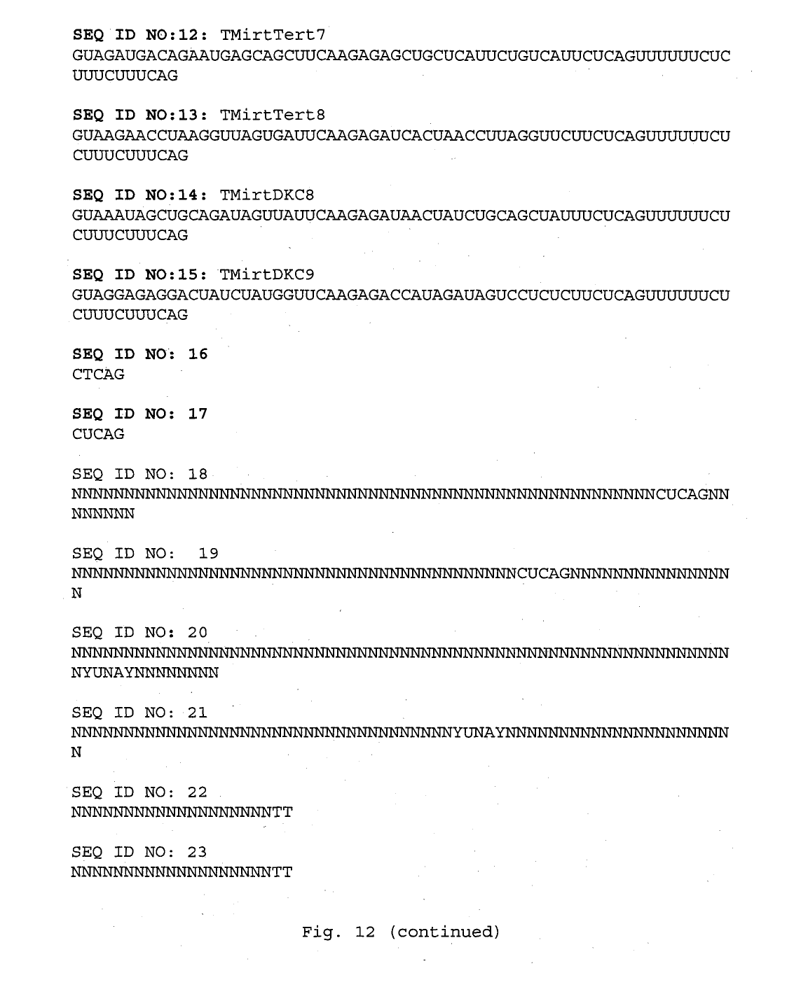

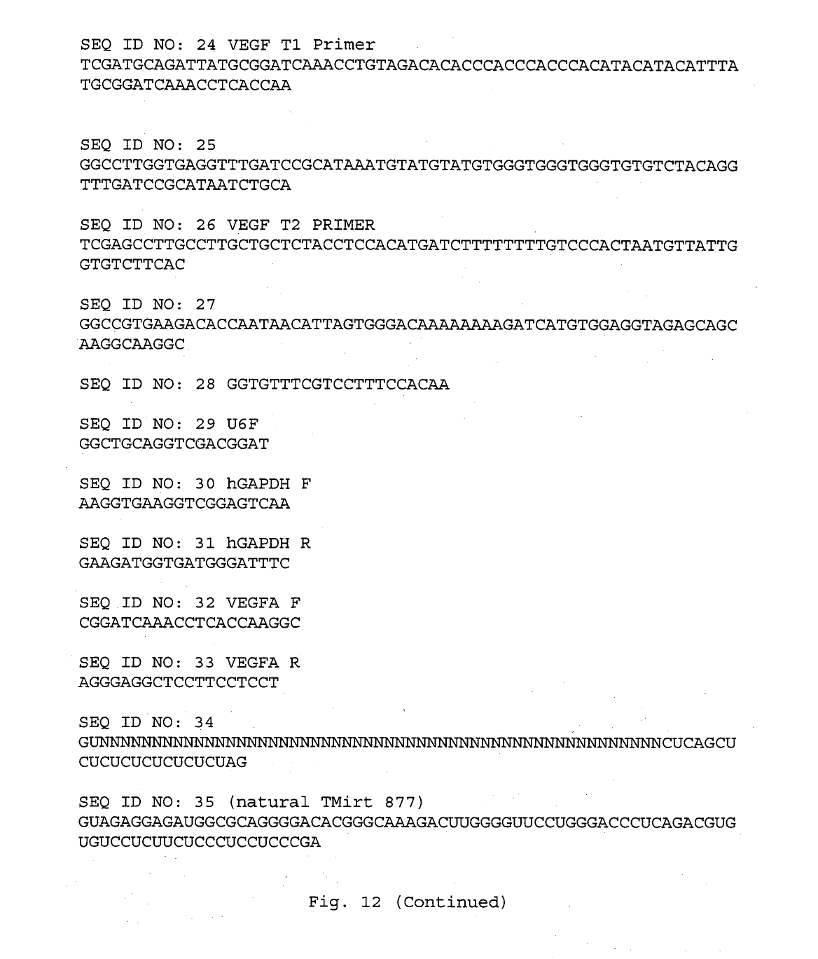

New TMirts targeting Firefly luciferase ORF with the guide strand in both 5′ arm and 3′ arm of the hairpin were also designed with a modified mirtron target identification algorithm without the preference for polypyrimidine or polypurine tracts (FIG. 9). These also resulted in significant knockdown of Firefly luciferase (FIG. 4e), validating the TMirts as an effective means to target ORFs of genes.

### Example 6

### TMirts Against VEGFA Results in Functional Knockdown of VEGFA Levels

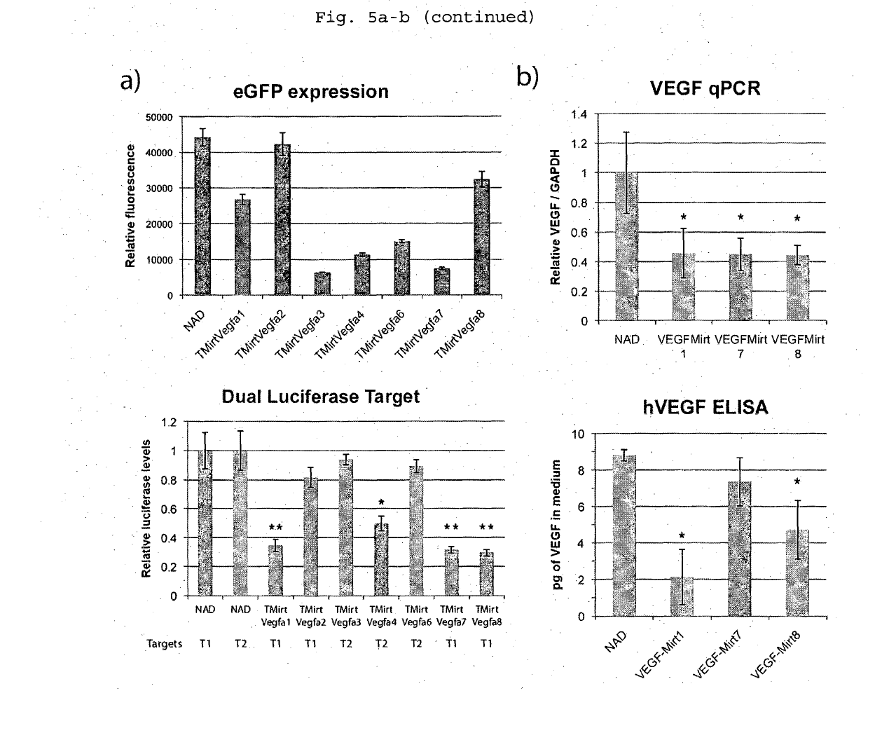

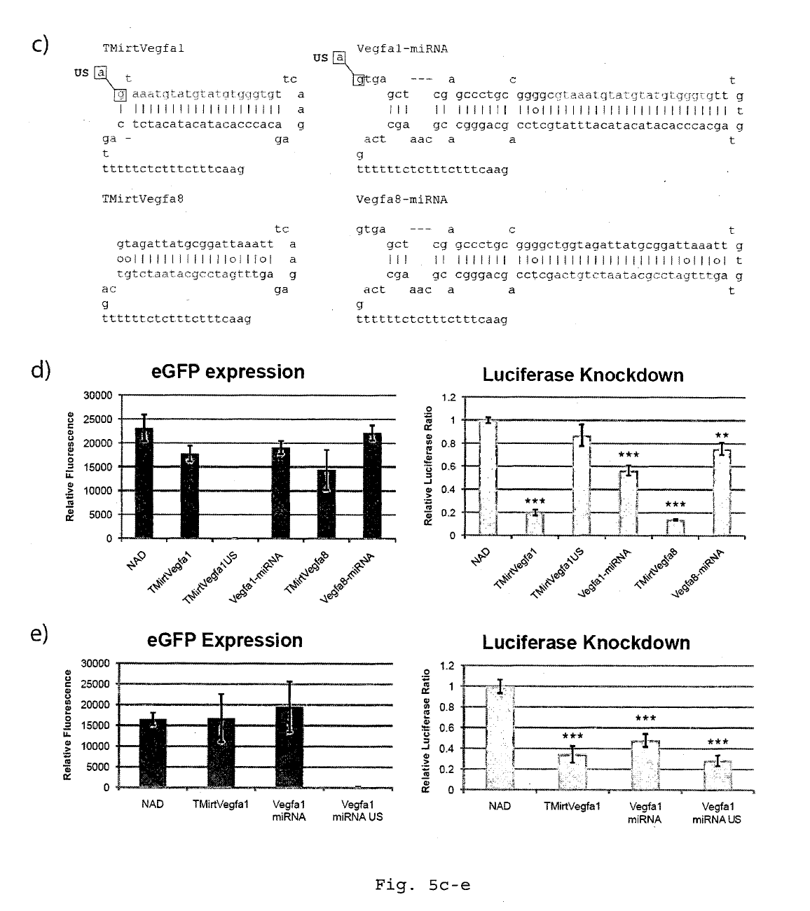

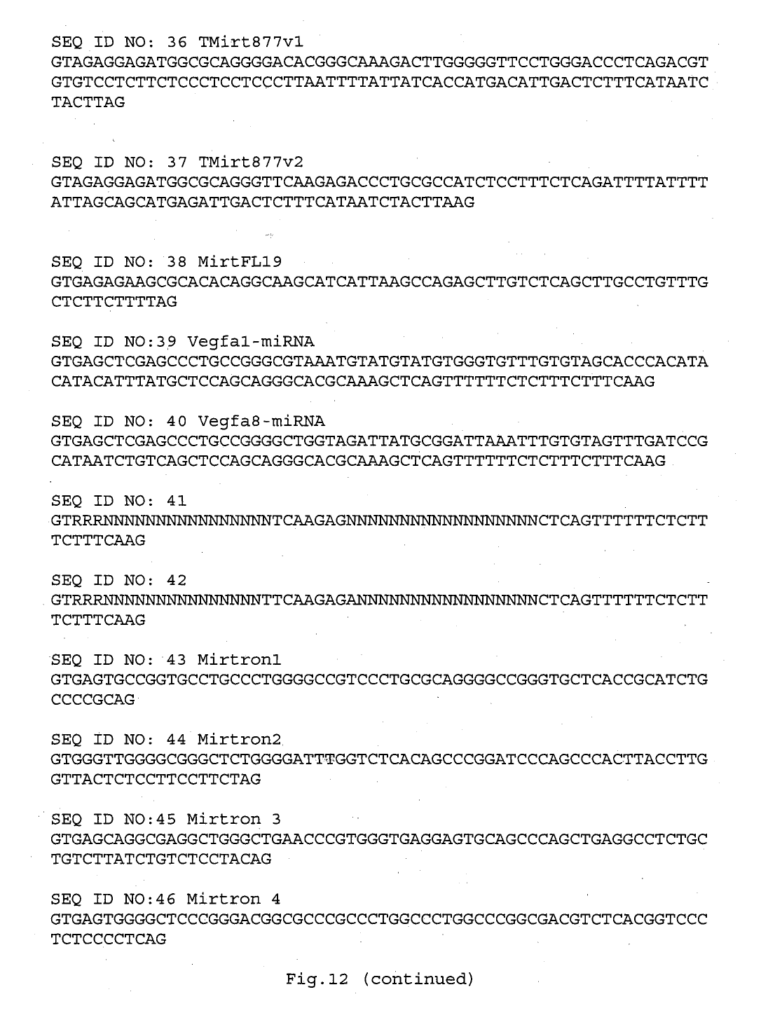

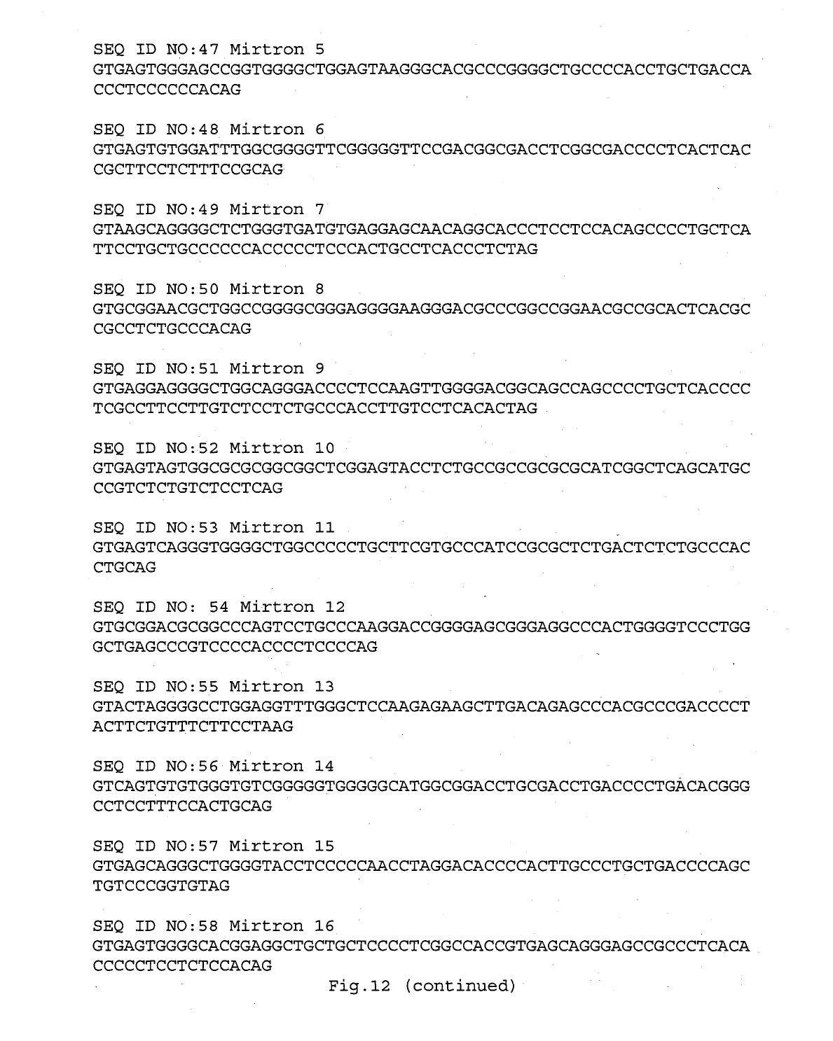

Tumour cells secrete VEGFA to promote angiogenesis. Efforts to target VEGFA signaling with siRNA and monoclonal antibody have gained clinical traction although one can only be cautiously optimistic about the weak effect on survival. The ability to target tumours concurrently from multiple angles may help improve the efficacy of therapeutics. VEGFA mirtrons can be one means to allow for co-delivery of a suicide gene with RNAi modalities. Thus, mirtrons targeted against VEGFA were designed and tested for knockdown using luciferase constructs first. All mirtrons spliced relatively well compared to NAD, but only designs 1, 4, 7 and 8 achieved luciferase knockdown (FIG. 5a, sequence in FIG. 10). Of the 4, 3 were chosen to validate in a functional assay with hypoxia-induced VEGF expression model. HEK293 cells were transfected with the TMirts and incubated in a hypoxic chamber for 24 hours. Subsequently, cell culture medium was collected and RNA harvested for quantitative RT-PCR. qRT-PCR results suggest that endogenous VEGFA mRNA was degraded by the mirtrons and subsequently, VEGFA secretion was also strongly inhibited by TMirtVegfa1 and less so with TMirtVegfa8 (FIG. 5b), thus demonstrating that TMirts against VEGFA can potently inhibit endogenous Vegfa secretion and thus can be a potential component in cancer gene therapeutics.

VEGFA TMirts are Superior to miRNA Mimics Designed with the Same Guide Strand

As pri-miRNA hairpins can also be nested within introns as miRNA-mimics and processed by Drosha, we sought to identify the differences between the two RNAi effectors. miRNA mimics with the guide sequences of VEGFA TMirts were designed based on the miR-106b cluster and inserted as an intron into peGFP-Mirt (FIG. 5c). Compared to the TMirts, the miRNA-mimics resulted in weaker knockdown at the same transfection ratios (FIG. 5d).

While TMirts are dependent on splicing for biogenesis, where mutation of the 5′ splice site (FIGS. 3c, 3g and 4d) abrogated splicing and knockdown, miRNA-mimics do not depend on splicing for biogenesis, as mutation of the 5′ splice site which abrogated splicing actually increased the knockdown of the luciferase target (FIG. 5e). This difference in the means of producing the pre-miRNA hairpin may influence the resultant homogeneity of the guide strands as shown by high throughput sequencing.

### TMirts can be Designed for Most Transcripts

To validate this approach of using TMirts for therapeutics, other TMirts against other proteins and RNAs of therapeutic interest were designed and tested, essentially the components of the telomerase complex—Tert1, DKC1 and TerC RNA. At least 2 mirtrons out of 10 designed against each target resulted in reasonable knockdown in luciferase constructs (FIG. 11), underlying the general applicability of TMirts for gene knockdown.

### Design Features for TMirts

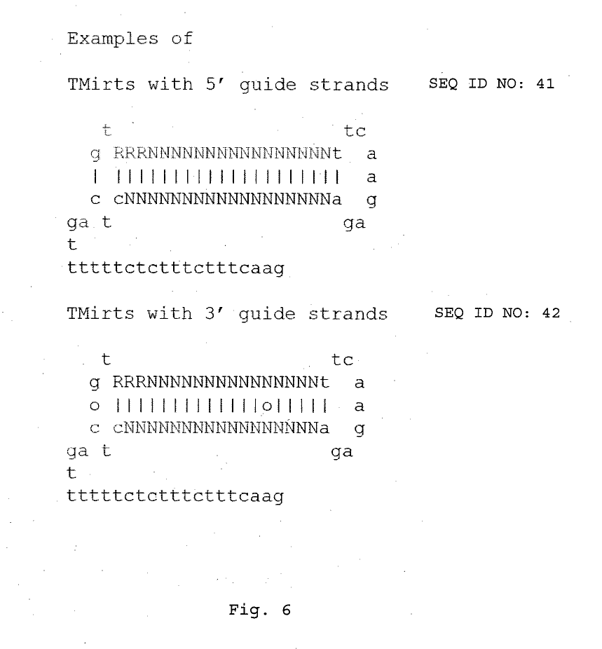

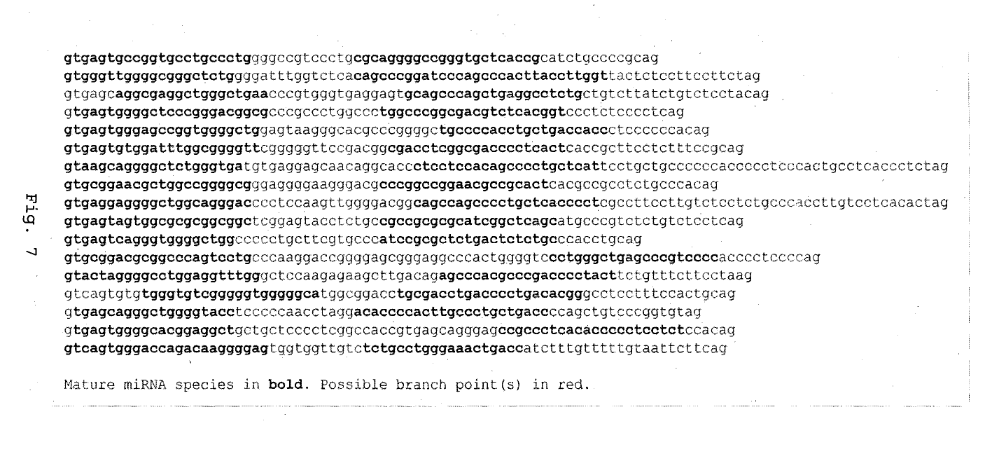

Based on small RNA sequencing of the TMirts, a few design principles to produce precise mature guide RNAs can be elucidated. The key features of the design involves 1) a canonical GU 5′ splice site followed by 3 purines, although splicing may still occur if one or two of the purines was substituted with pyrimidines, 2) a short hairpin to connect the 5′ arm guide/passenger strand to the 3′ arm passenger/guide strand, 3) designing the hairpin to end 1 nucleotide after the branch point A and 4) a strong branch point and polypyrimidine tract (FIG. 6).

### Applications

In summary, the disclosed tailed mirtrons are useful as alternative molecules for RNA interference (RNAi) that require minimal design constraints.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. An isolated nucleic acid molecule capable of binding a target nucleic acid sequence, wherein the isolated nucleic acid molecule comprises:
a. a stem loop structure comprising
i. a stem comprising a 5′ arm and a 3′ arm complementarily bound to each other, wherein the 5′ arm and the 3′ arm are connected to each other by a single stranded loop structure;
ii. a 5′ splice site;
iii. a nucleic acid sequence that is at least 50% complementary to a nucleotide sequence of the target nucleic acid sequence;
b. a tail sequence comprising
i. a 3′ splice site;
ii. a polypyrimidine-comprising sequence; and
c. a branch point sequence comprising a branch point, wherein the branch point sequence is located at the 3′ end of the 3′ arm of the stem loop structure, or up to 4 nucleotides upstream from the 3′ end of the 3′ arm of the stem loop structure, or up to 4 nucleotides downstream from the 5′ end of the tail sequence.

2. The isolated nucleic acid molecule according to claim 1, wherein the isolated nucleic acid molecule is asymmetric.

3. The isolated nucleic acid molecule according to claim 1, wherein the 5′ splice site is located at the 5′ end of the 5′ arm.

4. The isolated nucleic acid molecule according to claim 1, wherein the 5′ end of the stem loop comprises a 1 or 2 nucleotide 5′ overhang.

5. The isolated nucleic acid molecule according to claim 1, wherein the single stranded loop structure is 10 nucleotides or less.

6. The isolated nucleic acid molecule according to claim 1, wherein the 3′ splice site is located at the 3′ end of the tail sequence.

7. The isolated nucleic acid molecule according to claim 1, wherein the branch point is an adenosine.

8. The isolated nucleic acid molecule according to claim 1, wherein the polypyrimidine tract comprises of at least 70% of pyrimidine nucleotides.

9. The isolated nucleic acid molecule according to claim 1, wherein the polypyrimidine tract comprises 15 nucleotides or more.

10. The isolated nucleic acid molecule according to claim 1, wherein the 5′ and 3′ arm of the stem each comprises 29 nucleotides or less complementarily bound to each other.

11. The isolated nucleic acid molecule according to claim 10, wherein an A and U; G and C; or G and U are complementary bound to one another.

12. The isolated nucleic acid molecule according to claim 10, wherein the stem comprises up to 25% of nucleotides mismatch between the 5′ arm and the 3′ arm.

13. The isolated nucleic acid molecule according to claim 1, wherein the 5′ splice site is a GU splice site followed by 3 nucleotides selected from a group consisting of 3 purines; 2 purines and 1 pyrimidine, and 1 purine and 2 pyrimidines.

14. The isolated nucleic acid molecule according to claim 1, wherein the 5′ or the 3′ arm of the stem-loop structure is a guide strand.

15. The isolated nucleic acid molecule according to claim 14, wherein the guide strand comprises the nucleic acid sequence that is at least 50% complementary to a nucleotide sequence of the target nucleic acid sequence.

16. The isolated nucleic acid molecule according to claim 1, wherein the isolated nucleic acid is TMirt877v3.1 (SEQ ID NO: 1), TMir877v3.2 (SEQ ID NO: 2), DMPK TMirt5 (SEQ ID NO: 3), DMPKTMirt13 (SEQ ID NO: 4), TMirtFL19 (SEQ ID NO: 5), TMirtVEGF1 (SEQ ID NO: 6), TMirtVEGF4 (SEQ ID NO: 7), TMirtVEGF7 SEQ ID NO: 8), TMirtVEGF8 (SEQ ID NO: 9), TmirtTerC2 (SEQ ID NO: 10), TmirtTerC4 (SEQ ID NO: 11), TmirtTert7 SEQ ID NO: 12), TmirtTert8 (SEQ ID NO: 13), TmirtDKC8 (SEQ ID NO: 14) and TMirtDKC9 (SEQ ID NO: 15).

17. A DNA molecule encoding an RNA molecule comprising an isolated nucleic acid molecule capable of binding a target nucleic acid sequence, wherein the isolated nucleic acid molecule comprises:
a. a stem loop structure comprising
i. a stem comprising a 5′ arm and a 3′ arm complementarily bound to each other, wherein the 5′ arm and the 3′ arm are connected to each other by a single stranded loop structure;
ii. a 5′ splice site;
iii. a nucleic acid sequence that is at least 50% complementary to a nucleotide sequence of the target nucleic acid sequence;
b. a tail sequence comprising
i. a 3′ splice site;
ii. a polypyrimidine-comprising sequence; and
c. a branch point sequence comprising a branch point, wherein the branch point sequence is located at the 3′ end of the 3′ arm of the stem loop structure, or up to 4 nucleotides upstream from the 3′ end of the 3′ arm of the stem loop structure, or up to 4 nucleotides downstream from the 5′ end of the tail sequence.

18. A vector comprising an isolated nucleic acid molecule capable of binding a target nucleic acid sequence, wherein the isolated nucleic acid molecule comprises:
a. a stem loop structure comprising
iv. a stem comprising a 5′ arm and a 3′ arm complementarily bound to each other, wherein the 5′ arm and the 3′ arm are connected to each other by a single stranded loop structure;
v. a 5′ splice site;
vi. a nucleic acid sequence that is at least 50% complementary to a nucleotide sequence of the target nucleic acid sequence;
b. a tail sequence comprising
i. a 3′ splice site;
ii. a polypyrimidine-comprising sequence; and
c. a branch point sequence comprising a branch point, wherein the branch point sequence is located at the 3′ end of the 3′ arm of the stem loop structure, or up to 4 nucleotides upstream from the 3′ end of the 3′ arm of the stem loop structure, or up to 4 nucleotides downstream from the 5′ end of the tail sequence.

19. A pharmaceutical composition comprising an isolated nucleic acid molecule capable of binding a target nucleic acid sequence, wherein the isolated nucleic acid molecule comprises:
a. a stem loop structure comprising
i. a stem comprising a 5′ arm and a 3′ arm complementarily bound to each other, wherein the 5′ arm and the 3′ arm are connected to each other by a single stranded loop structure;
ii. a 5′ splice site;
iii. a nucleic acid sequence that is at least 50% complementary to a nucleotide sequence of the target nucleic acid sequence;
b. a tail sequence comprising
i. a 3′ splice site;
ii. a polypyrimidine-comprising sequence; and
c. a branch point sequence comprising a branch point, wherein the branch point sequence is located at the 3′ end of the 3′ arm of the stem loop structure, or up to 4 nucleotides upstream from the 3′ end of the 3′ arm of the stem loop structure, or up to 4 nucleotides downstream from the 5′ end of the tail sequence.

20. A method of treating a disease comprising administering a pharmaceutical composition comprising an isolated nucleic acid molecule capable of binding a target nucleic acid sequence, wherein the isolated nucleic acid molecule comprises:
a. a stem loop structure comprising
i. a stem comprising a 5′ arm and a 3′ arm complementarily bound to each other, wherein the 5′ arm and the 3′ arm are connected to each other by a single stranded loop structure;
ii. a 5′ splice site;
iii. a nucleic acid sequence that is at least 50% complementary to a nucleotide sequence of the target nucleic acid sequence;
b. a tail sequence comprising
i. a 3′ splice site;
ii. a polypyrimidine-comprising sequence; and
c. a branch point sequence comprising a branch point, wherein the branch point sequence is located at the 3′ end of the 3′ arm of the stem loop structure, or up to 4 nucleotides upstream from the 3′ end of the 3′ arm of the stem loop structure, or up to 4 nucleotides downstream from the 5′ end of the tail sequence, to a patient in need of gene therapy.
